(19) United States
(12) Patent Application Publication
(10) Pub. No.: US 2014/0294142 A1
(43) Pub. Date: Oct. 2, 2014

(54) X-RAY IMAGING DEVICE AND METHOD OF CONTROLLING THE SAME

(71) Applicant: SAMSUNG ELECTRONICS CO., LTD., Suwon-si (KR)

(72) Inventor: Jin Ho CHOI, Anyang-si (KR)

(73) Assignee: SAMSUNG ELECTRONICS CO., LTD., Suwon-si (KR)

(21) Appl. No.: 14/224,567

(22) Filed: Mar. 25, 2014

(30) Foreign Application Priority Data
Mar. 28, 2013 (KR) 10-2013-0033431
Jan. 3, 2014 (KR) 10-2014-0000901

(51) Int. Cl. A61B 6/00

(52) U.S. Cl. CPC A61B 6/54; A61B 6/502
USPC 378/37; 378/91

(57) ABSTRACT

An X-ray imaging device and a method for controlling the same generates radiant heat to elevate a temperature of an area of the X-ray imaging device contacting the breast and thereby reduces patient's discomfort, and controls a temperature to maintain the temperature.

The X-ray imaging device includes an X-ray source to generate X-rays and emit the X-rays to an object, an X-ray detector to detect X-rays transmitted through the object and convert the X-rays into an electrical signal, an object contact area mounted on the X-ray detector a compression paddle to compress the object disposed on the object contact area, a heat source to generate heat and transfer the heat to at least one of the object contact area and the compression paddle, and an insulation member formed between the object contact area and the X-ray detector to block transfer of heat to the X-ray detector.

### CROSS-REFERENCE TO RELATED APPLICATIONS

This application claims the benefit of Korean Patent Application No. 10-2013-0033431 filed on Mar. 28, 2013 in the Korean Intellectual Property Office and Korean Patent Application No. 10-2014-0000901 filed on Jan. 3, 2014 in the Korean Intellectual Property Office, the disclosures of which are incorporated herein by reference.

### BACKGROUND

1. Field

One or more embodiments relate to an X-ray imaging device for forming an X-ray image by radiating X-rays to an object, in particular, a breast, and a method of controlling the same.

2. Description of the Related Art

An X-ray imaging device acquires an inner image of an object by radiating X-rays to the object and analyzing X-rays having transmitted through the object. Because transmittance of X-rays varies depending on a material constituting the object, an inner structure of the object is imaged by detecting a level or intensity of X-rays having transmitted through the object.

Mammography is an X-ray imaging method for examining a breast as an object. It may be necessary to perform X-ray imaging in a state in which a breast disposed between an X-ray source and an X-ray detector is compressed by a compression paddle so as to obtain an accurate and clear X-ray image of an inner structure of the breast, because mammary gland tissues and fat tissues are grown in the breast.

The X-ray imaging device and a scan room in which the X-ray imaging device is disposed are kept at a low temperature, causing a patient to feel cold. For this reason, there is a problem in that a patient suffers from pain by compression and discomfort due to coldness when the breast of the patient who takes off her top is compressed by a cold compression paddle.

Meanwhile, the breast of the patient is compressed by the compression paddle while the breast of the patient is placed on a carbon sheet present in an upper part of an X-ray detector.

### SUMMARY

The foregoing described problems may be overcome and/or other aspects may be achieved by one or more embodiments of an X-ray imaging device which may generate radiant heat to elevate a temperature of an area of the X-ray imaging device contacting the breast and thereby may reduce the patient's discomfort, and may automatically control a temperature to maintain the temperature without separate operation of a user and a method for controlling the same.

In addition, the foregoing described problems may be overcome and/or other aspects may be achieved by one or more embodiments of an X-ray imaging device which may automatically sterilize the portion of the X-ray imaging device contacting the breast when the X-ray imaging device is not used, and thereby may maintain cleanliness while possibly not increasing work fatigue, and a method of controlling the same.

Additional aspects and/or advantages of one or more embodiments will be set forth in part in the description which follows and, in part, will be apparent from the description, or may be learned by practice of one or more embodiments of disclosure. One or more embodiments are inclusive of such additional aspects.

In accordance with one or more embodiments, an X-ray imaging device for forming an X-ray image of a breast may include an X-ray source to generate X-rays and emit the X-rays to an object, an X-ray detector to detect X-rays having transmitted through the object and convert the X-rays into an electric signal, an object contact area mounted in an upper part of the X-ray detector, the object contact area contacting the object, a compression paddle to compress the object disposed on the object contact area, a heat source to generate heat and transfer the heat to at least one of the object contact area and the compression paddle, and an insulation member formed between the object contact area and the X-ray detector to block transfer of heat to the X-ray detector.

The heat source may be mounted in a lower part of the X-ray source.

The insulation member may include a film to block transfer of heat.

The insulation member may include a coating layer coated with a material blocking transfer of heat.

The insulation member may not block transmission of X-rays.

The X-ray imaging device may further include a temperature sensor to sense a temperature of the object contact area, wherein the temperature sensor is disposed outside of the X-ray detector.

The X-ray imaging device may further include a controller to control an amount of heat generated by the heat source, based on the temperature sensed by the temperature sensor.

The controller may decrease an amount of heat generated by the heat source when the temperature sensed by the temperature sensor is higher than a predetermined upper limit and may increase the amount of heat generated by the heat source when the temperature sensed by the temperature sensor is lower than a predetermined lower limit.

The controller may cut off power to the heat source to stop heat generation, when X-ray imaging starts.

The X-ray imaging device may further include an ultraviolet light generator to emit ultraviolet light to at least one of the object contact area and the compression paddle.

The controller may turn off the ultraviolet light generator when X-ray imaging starts and may turn on the ultraviolet light generator when X-ray imaging finishes.

The heat source may generate radiant heat, wherein the radiant heat may include infrared light.

The object contact area may include carbon.

The insulation member may block infrared light having a wavelength range of about 0.75 μm to about 1 mm and may not block X-rays having a wavelength range of about 0.001 nm to about 10 nm.

In accordance with one or more embodiments, an X-ray imaging device of producing an X-ray image may include an X-ray tube to generate X-rays and emit the X-rays to an object, an X-ray detector to detect X-rays transmitting the object and convert the X-rays into an electrical signal, an object contact area being mounted in an upper part of the X-ray detector and contacting the object, a ultraviolet light generator to emit ultraviolet light to the object contact area and a controller to control the ultraviolet light generator.

The ultraviolet light generator may be disposed adjacent to the X-ray tube.

The X-ray imaging device may further include a collimator to control an emission area of the ultraviolet light emitted from the ultraviolet light generator and the ultraviolet light generator may be provided inside of the collimator.

The ultraviolet light generator may be disposed adjacent to a visible light source provided inside of the collimator.

The collimator may include at least one shutter to shield ultraviolet light and a shutter driving unit to move the shutter.

The controller may perform a control operation such that the shutter driving unit may move the shutter to a position corresponding to a predetermined emission area of ultraviolet light.

Position and area of the emission area of ultraviolet light may be set to correspond to position and area of the object contact area.

The controller may determine an idle period in which X-ray imaging is not performed on the object and perform a control operation such that the ultraviolet light generator is turned on during the idle period.

The X-ray imaging device may further include an object sensor to sense the object present on the object contact area.

The controller may perform a control operation such that the ultraviolet light generator is turned on when an output signal of the object sensor indicates that the object is not present on the object contact area.

The object sensor may include at least one sensor selected from the group consisting of an infrared sensor, an ultrasonic sensor and a touch sensor.

The X-ray imaging device may further include a compression paddle that may be removably mounted on a frame to support the X-ray tube and the X-ray detector, the compression paddle that may compress the object disposed on the object contact area, and a paddle sensor that may sense the compression paddle mounted on the frame.

The controller may perform a control operation such that the ultraviolet light generator may be turned on when an output signal of the paddle sensor indicates that the compression paddle is not mounted.

The controller may perform a control operation such that the ultraviolet light generator is turned on, based on a work list indicating a list of X-ray imaging.

The X-ray imaging device may further include a compression paddle that may be removably mounted on a frame to support the X-ray tube and the X-ray detector, the compression paddle that may compress the object disposed on the object contact area, and a paddle sensor that may sense the compression paddle mounted on the frame.

The controller may perform a control operation such that the ultraviolet light generator is turned on when the output signal of the object sensor indicates that the object is not present on the object contact area and the output signal of the paddle sensor indicates that the compression paddle is not mounted.

The controller may perform a control operation such that the ultraviolet light generator is turned on when the output signal of the object sensor indicates that the object is not present on the object contact area and X-ray imaging is not present in the work list indicating a list of X-ray imaging.

The controller may perform a control operation such that the ultraviolet light generator is turned on when an output signal of the paddle sensor indicates that the compression paddle is not mounted and X-ray imaging is not present in the work list indicating a list of X-ray imaging.

The X-ray imaging device may further include a compression paddle that may be removably mounted on the frame to support the X-ray tube and the X-ray detector, the compression paddle that may compress the object disposed on the object contact area, and a heat source to transfer heat to at least one of the object contact area and the compression paddle.

The X-ray imaging device may further include an insulation member formed between the object contact area and the X-ray detector and possibly blocking transfer of heat to the X-ray detector.

The controller may detect an idle period in which X-ray imaging is not performed on the object and may perform a control operation such that the heat source is turned on during the idle period.

The X-ray imaging device may further include a paddle sensor to sense the compression paddle mounted on the frame and an object sensor to sense the object present on the object contact area.

The controller may perform a control operation such that the heat source is turned off and the ultraviolet light generator is turned on when an output signal of the object sensor indicates that the object is not present on the object contact area and an output signal of the paddle sensor indicates that the compression paddle is not mounted.

The controller may perform a control operation such that the heat source is turned on and the ultraviolet light generator is turned off when the output signal of the object sensor indicates that the object is not present on the object contact area and the output signal of the paddle sensor indicates that the compression paddle is mounted.

The collimator may be mounted in a lower part of the X-ray tube and may control an emission area of X-rays emitted from the X-ray tube.

In accordance with one or more embodiments, a method of controlling an X-ray imaging device to produce an X-ray image may include supplying power to a heat source mounted on the X-ray imaging device to generate heat, blocking transfer of the generated heat to an X-ray detector using an insulation member formed on a surface of the X-ray detector, and cutting-off power to the heat source to stop heat generation when X-ray imaging starts.

The heat source may be mounted at a position enabling transfer of heat to a compression paddle and an object contact area, as portions of the X-ray imaging device contacting the object.

The heat source may transfer heat by radiation.

The method may further include sensing a temperature of the compression paddle or the object contact area.

The method may further include controlling an amount of heat generated by the heat source, based on the sensed temperature.

The controlling may include increasing the amount of heat generated by the heat source when the sensed temperature is lower than a predetermined lower limit and decreasing the amount of heat generated by the heat source when the sensed temperature is higher than a predetermined upper limit.

The method may further include emitting ultraviolet light to the compression paddle and the object contact area when X-ray imaging finishes.

In accordance with one or more embodiments, a method of controlling an X-ray imaging device may include determining whether or not a current stage is an idle period in which X-ray imaging is not performed, performing a control operation such that a position of a collimator to control an emission area of X-rays corresponds to a predetermined emission area of ultraviolet light when the current stage is determined to be the idle period, and turning on an ultraviolet light generator that may be mounted in the collimator to possibly sterilize an object contact area that may contact an object.

The determining may include sensing an object present on the object contact area and determining the current stage to be the idle period when the object is not sensed.

The determining may include sensing a compression paddle mounted on a frame to support an X-ray tube and an X-ray detector and determining the current stage to be the idle period when the compression paddle is not sensed.

The determining may include analyzing a work list indicating a list of X-ray imaging and determining the current stage to be the idle period when remaining X-ray imaging is not present in the work list.

In accordance with one or more embodiments, an electro-magnetic radiation imaging device of producing an electro-magnetic radiation image may comprise an electro-magnetic radiation source to generate electro-magnetic radiation and emit the electro-magnetic radiation to an object; an electro-magnetic radiation detector to detect electro-magnetic radiation transmitted through the object and to convert the electro-magnetic radiation into an electrical signal; an object contact area mounted in an upper part of the electro-magnetic radiation detector, the object contact area contacting the object; and an auxiliary radiation generator mounted on the imaging device.

The electro-magnetic radiation imaging device may further comprise an insulation member formed between the object contact area and the electro-magnetic radiation detector, the insulation member blocking transfer of heat to the electro-magnetic radiation detector, wherein the auxiliary radiation generator comprises a heat source to generate heat and transfer the heat to the object contact area.

The electro-magnetic radiation imaging device may further comprise a compression paddle to compress the object disposed on the object contact area, wherein the auxiliary radiation generator further comprises an ultraviolet light generator to emit ultraviolet light to at least one of the object contact area and the compression paddle.

The electro-magnetic radiation imaging device may further comprise a controller to control the auxiliary radiation generator.

The controller cuts off power to the heat source to stop heat generation when electro- magnetic radiation imaging starts.

The electro-magnetic radiation imaging device may further comprise an object sensor to sense the object present on the object contact area.

The controller turns on the ultraviolet light generator when an output signal of the object sensor indicates that the object is not present on the object contact area.

The object sensor comprises one sensor selected from the group consisting of an infrared sensor, an ultrasonic sensor and a touch sensor.

The compression paddle is removably mounted, and the electro-magnetic radiation imaging device further comprises a paddle sensor that may sense the compression paddle mounted on the frame.

The controller turns on the ultraviolet light generator when an output signal of the paddle sensor indicates that the compression paddle is not mounted.

The controller turns on the ultraviolet light generator based on a work list indicating a list of electro-magnetic radiation imaging.

### BRIEF DESCRIPTION OF THE DRAWINGS

These and/or other aspects will become apparent and more readily appreciated from the following description of embodiments, taken in conjunction with the accompanying drawings of which:

FIG. 1 is a view illustrating an outer appearance of an X-ray imaging device used for breast X-ray imaging;

FIG. 2 is a sectional view illustrating an inner structure of a breast;

FIG. 3 is a graph showing attenuation coefficients of components constituting the breast;

FIG. 4 is a block diagram illustrating an X-ray imaging device according to one or more embodiments;

FIG. 5A is a sectional view illustrating an inner structure of an X-ray source;

FIG. 5B is a perspective view illustrating a configuration of an X-ray detector;

FIG. 6 is a view illustrating an outer appearance of an X-ray imaging device according to one or more embodiments;

FIG. 7 is a view illustrating another outer appearance of an X-ray imaging device according to one or more embodiments;

FIG. 8 is a block diagram illustrating an X-ray imaging device according to or more embodiments further including a temperature sensor;

FIG. 9 is a view illustrating an outer appearance of an X-ray imaging device according to or more embodiments further including a temperature sensor;

FIG. 10 is a block diagram illustrating a detailed configuration of a controller in an X-ray imaging device according to one or more embodiments;

FIG. 11 is a block diagram illustrating an X-ray imaging device according to or more embodiments further including an ultraviolet light generator;

FIGS. 12A and 12B are views illustrating an outer appearance of an X-ray imaging device according to or more embodiments further including an ultraviolet light generator;

FIG. 13 is a block diagram illustrating an X-ray imaging device according to one or more embodiments;

FIG. 14 is a view illustrating an outer appearance of an X-ray imaging device according to one or more embodiments;

FIG. 15 is a view illustrating an example of a collimator included in an X-ray imaging device according to one or more embodiments;

FIG. 16 is a view illustrating an indication operation of an X-ray imaging area by a visible light source provided in a collimator according to one or more embodiments;

FIG. 17 is a view illustrating a control operation of emission areas of X-rays emitted from an X-ray tube, by a shutter according to one or more embodiments;

FIG. 18 is a view illustrating a control operation of emission areas of ultraviolet light emitted from an ultraviolet light generator, by a shutter according to one or more embodiments;

FIG. 19 is a block diagram illustrating an X-ray imaging device according to one or more embodiments further including an object sensor;

FIG. 20 is a view illustrating an outer appearance of an X-ray imaging device according to one or more embodiments further including an object sensor;

FIG. 21 is a block diagram illustrating a configuration of an X-ray imaging device according to one or more embodiments further including a paddle sensor;

FIG. 22 is a view illustrating an outer appearance of the configuration of an X-ray imaging device according to one or more embodiments further including a paddle sensor;

FIG. 23 is a block diagram illustrating a receiving operation of a work list from a server, in an X-ray imaging device according to one or more embodiments;

FIG. 24 is a block diagram illustrating a configuration of an X-ray imaging device according to one or more embodiments further including an object sensor and a paddle sensor;

FIG. 25 is a view illustrating an outer appearance of an X-ray imaging device according to one or more embodiments further including an object sensor and a paddle sensor;

FIG. 26 is a block diagram illustrating a control operation of generation of ultraviolet light based on an output signal of an object sensor and a work list, in an X-ray imaging device according to one or more embodiments;

FIG. 27 is a block diagram illustrating a control operation of generation of ultraviolet light, based on an output signal of a paddle sensor and a work list, in an X-ray imaging device according to one or more embodiments;

FIG. 28 is a block diagram illustrating a control operation of generation of ultraviolet light, based on an output signal of an object sensor, the output signal of a paddle sensor and a work list, in an X-ray imaging device according to one or more embodiments;

FIG. 29 is a block diagram illustrating a configuration of an X-ray imaging device according to one or more embodiments further including a heat source unit;

FIG. 30 is a flowchart illustrating a method of controlling an X-ray imaging device according to one or more embodiments;

FIG. 31 is a flowchart illustrating a method of controlling an X-ray imaging device according to one or more embodiments further including ultraviolet light sterilization;

FIG. 32 is a flowchart illustrating a method of controlling an X-ray imaging device according to one or more embodiments;

FIG. 33 is a flowchart illustrating a control process of generation of ultraviolet light according to presence of an object, in a method of controlling an X-ray imaging device according to one or more embodiments;

FIG. 34 is a flowchart illustrating a control process of generation of ultraviolet light according to presence of a compression paddle, in a method of controlling an X-ray imaging device according to one or more embodiments;

FIG. 35 is a flowchart illustrating a control process of generation of ultraviolet light through analysis of a work list, in a method of controlling an X-ray imaging device according to one or more embodiments;

FIG. 36 is a flowchart illustrating a control process of generation of ultraviolet light according to presence of an object and a compression paddle, in a method of controlling an X-ray imaging device according to one or more embodiments;

FIG. 37 is a flowchart illustrating a control process of generation of ultraviolet light based on presence of an object and a work list, in a method of controlling an X-ray imaging device according to one or more embodiments;

FIG. 38 is a flowchart illustrating a control process of generation of ultraviolet light based on presence of a compression paddle and a work list, in a method of controlling an X-ray imaging device according to one or more embodiments; and

FIG. 39 is a flowchart illustrating a control process of generation of ultraviolet light based on presence of an object and a compression paddle and a work list, in the method of controlling an X-ray imaging device according to one or more embodiments.

### DETAILED DESCRIPTION

Reference will now be made in detail to one or more embodiments, illustrated in the accompanying drawings, wherein like reference numerals refer to like elements throughout. In this regard, embodiments of the present invention may be embodied in many different forms and should not be construed as being limited to embodiments set forth herein, as various changes, modifications, and equivalents of the systems, apparatuses and/or methods described herein will be understood to be included in the invention by those of ordinary skill in the art after embodiments discussed herein are understood. Accordingly, embodiments are merely described below, by referring to the figures, to explain aspects of the present invention.

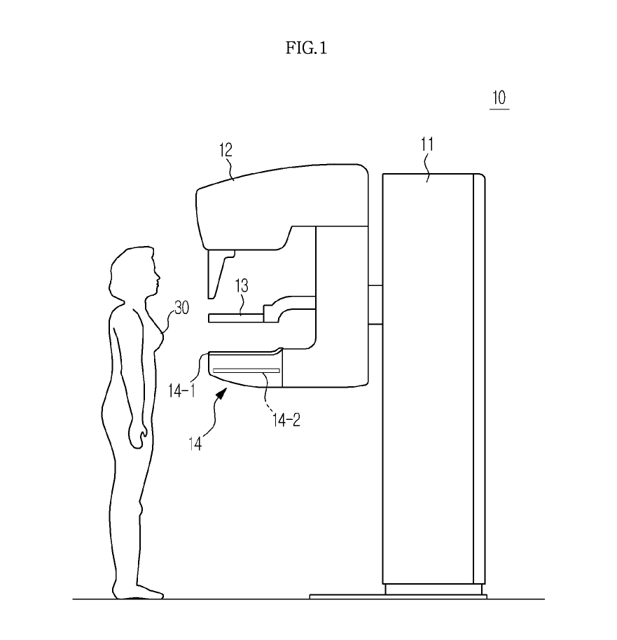

FIG. 1 is a view illustrating an external appearance of an X-ray imaging device used for breast X-ray imaging.

The X-ray imaging device 10 used for breast X-ray imaging has a structure specialized for imaging a breast, unlike a general X-ray imaging device. Specifically, as shown in FIG. 1, an X-ray source 12 and an X-ray detector assembly 14 are mounted on a gantry 11, X-rays are emitted to an object 30 disposed between the X-ray source 12 and the X-ray detector assembly 14 and X-rays transmitted through the object 30 are detected to obtain an X-ray image of the object 30. The object 30 is a breast of a patient.

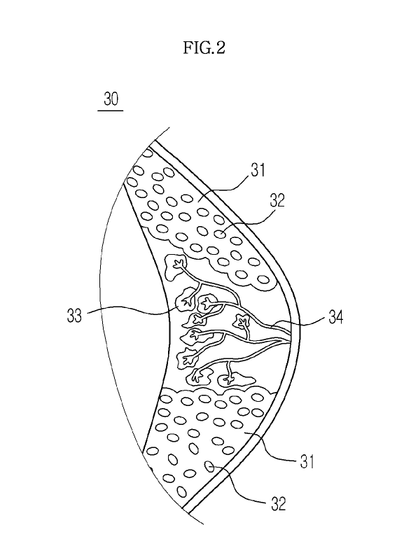

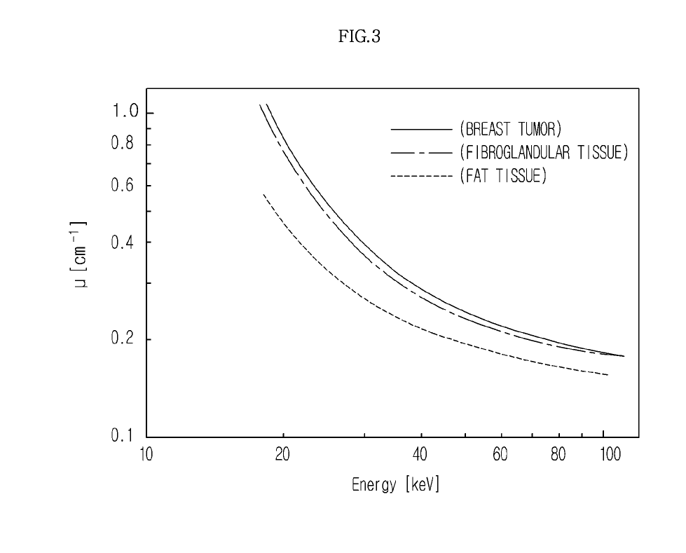

Referring to FIG. 1, the X-ray imaging device 10 further includes a compression paddle 13 disposed between the X-ray source 12 and the X-ray detector assembly 14. The compression paddle 13 compresses the object 30 placed on an object contact area 14-1. Hereinafter, operations of the compression paddle will be described in detail with reference to FIGS. 2 and 3.

FIG. 2 is a sectional view illustrating an inner structure of a breast and FIG. 3 is a graph showing attenuation coefficients of components constituting the breast.

Referring to FIG. 2, tissue of the breast 30 includes a fibrous tissue 31 surrounding the breast 30 and maintaining a shape thereof, a fatty tissue 32 present throughout the breast, a mammary gland tissue 33 to produce milk and a lactiferous duct tissue 34 providing a passage of the milk. A tissue associated with production and supply of milk, such as the mammary gland tissue 33 or the lactiferous duct tissue 34 among these tissues, is referred to as fibroglandular tissue of the breast.

An attenuation coefficient is data indicating an attenuation level of X-rays when X-rays pass through an object. Imaging an inner structure of the object is possible, since an attenuation coefficient is varied according to components constituting the inner structure of the object. FIG. 3 shows attenuation coefficients of breast tumor, fibroglandular tissue and fat tissue, as components constituting the breast, according to energy regions. As shown in FIG. 3, a difference in attenuation coefficient between the components constituting the breast is not great. As shown in FIG. 2, this is because the breast consists entirely of soft tissue. In order to obtain a clear X-ray image, the breast is compressed using the compression paddle 13. Furthermore, when the breast is compressed, a thickness of the object is decreased and an X-ray exposure dose is thus decreased.

Referring to FIG. 1 again, for X-ray imaging, when the breast 30 as the object is placed on the object contact area 14-1, the compression paddle 13 compresses the breast 30, and when the X-ray source 12 emits towards the compressed breast 30, an X-ray detector 14-2 detects X-rays transmitted through the breast 30 to obtain an X-ray image of the breast.

Some elements constituting the X-ray imaging device 10 are sensitive to temperature. In particular, the X-ray detector 14-2 is sensitive to temperature since it includes a semiconductor element. When a temperature suitable for characteristics of the semiconductor element is not maintained, the element does not function normally and causes errors. Accordingly, a scan room in which the X-ray imaging device 10 is disposed and the X-ray imaging device 10 maintain a predetermined temperature suitable for characteristics of the element.

However, the temperature suitable for characteristics of the element is low to an extent that human feels cold. A patient who entirely or partially takes off her top may feel cold during imaging and suffers from compression pain as well as discomfort caused by coldness, because an area for breast compression contacting the patient is cold.

Accordingly, the X-ray imaging device according to one or more embodiments may include a heat source to increase a temperature of the area of the X-ray imaging device contacting the breast and thereby minimize patient discomfort. Hereinafter, a detailed configuration and operation of the X-ray imaging device according to one or more embodiments will be described.

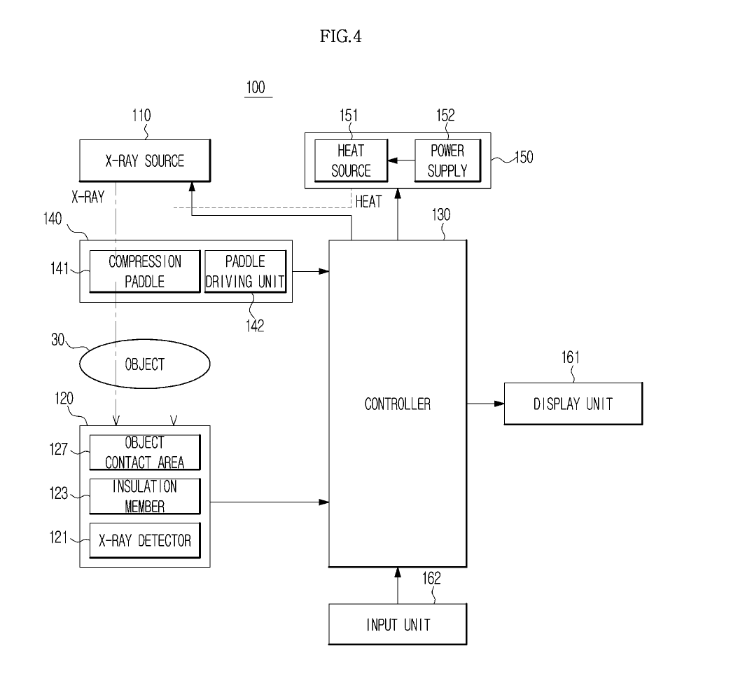

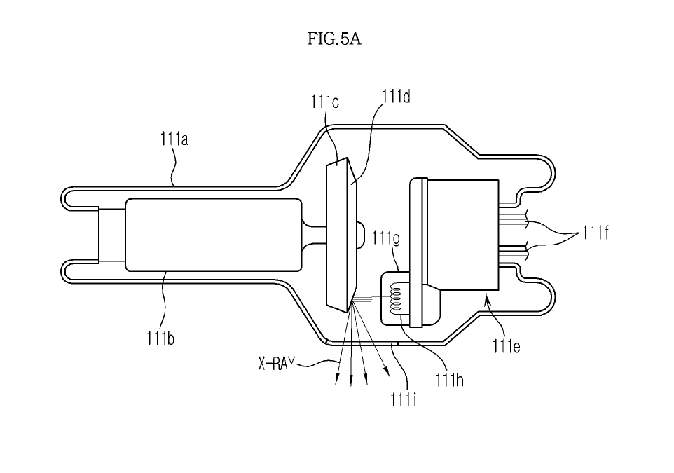

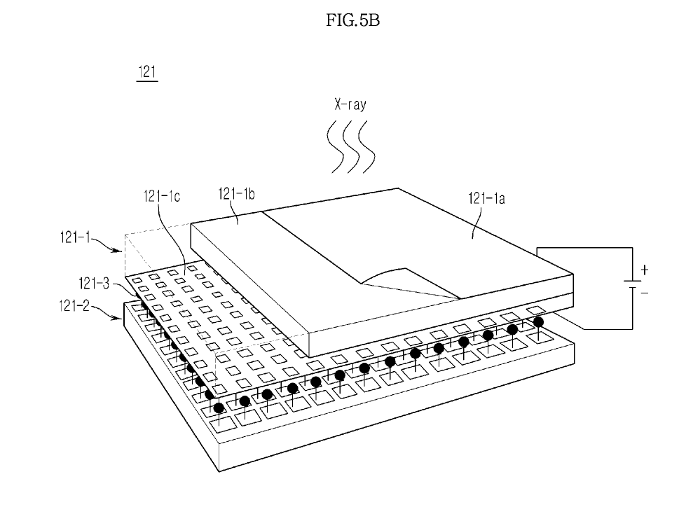

FIG. 4 is a block diagram illustrating a controlled configuration of the X-ray imaging device according to one or more embodiments, FIG. 5A is a sectional view illustrating an inner structure of an X-ray source and FIG. 5B is a perspective view illustrating a configuration of an X-ray detector.

Referring to FIG. 4, the X-ray imaging device 100 may include an X-ray source 110 to generate X-rays and emit the same to the object 30, an X-ray detector assembly 120 to detect X-rays having transmitted through the object 30, a compression paddle assembly 140 to compress the object 30, a heat source unit 150 to emit heat to the compression paddle 141 and the object contact area 127, a controller 130 to control an operation of the X-ray imaging device 100, a display unit 161 to display an X-ray image and an input unit 162 to input a control instruction associated with the operation of the X-ray imaging device 100.

Although X-rays are illustrated as an example of electromagnetic radiation, other types of electromagnetic radiation may be applied to an embodiment of the present disclosure.

The X-ray source 110 receives power from a power supply and generates X-rays. X-ray energy is controlled by tube voltage and tube current, and an intensity or dose of X-rays is controlled by X-ray exposure time.

Referring to FIG. 5A, the X-ray source 110 may include an X-ray tube 111 to generate X-rays and the X-ray tube 111 may be implemented with a two-electrode vacuum tube including an anode 111c and a cathode 111e. The cathode 111e includes a filament 111h and a focusing electrode 111g to focus electrons, and the focusing electrode 111g is also called a “focusing cup”

A glass tube 111a is evacuated to a high level of about 10 mmHg and the filament 111h of the cathode is heated to a high temperature to generate thermoelectrons. An example of the filament 111h is a tungsten filament. The filament 111h is heated by applying current to a wire 111f connected to the filament.

The anode 111c is made of copper, a target material 111d is applied or disposed opposite to the cathode 111e, and the target material 111d may be a high-resistant material such as Cr, Fe, Co, Ni, W or Mo. As melting point of the target material increases, a focal spot size decreases. The focal spot means an effective focal spot. In addition, the target material is inclined at a predetermined angle. As inclination angle decreases, focal spot size decreases.

When high voltage is applied between the cathode 111e and the anode 111c, the thermoelectrons accelerate and collide with the target material 111g of the anode to generate X-rays. The generated X-rays are emitted to the outside through a window 111i and a material for the window 111i may be a beryllium (Be) thin film. A filter is loaded on a front or rear surface of the window 111i to filter a specific energy band of X-rays.

The target material 111d is rotated by a rotor 111b. When the target material 111d is rotated, heat accumulation is increased to 10-fold or more and focal spot size decreases, as compared to when the target is fixed.

The voltage applied between the cathode 111e and the anode 111c of the X-ray tube 111 is referred to as a “tube voltage” and a level of the voltage is represented as kvp. As tube voltage increases, a velocity of thermoelectrons increases and as a result, energy of X-rays (energy of photons) generated by collision of thermoelectrons with the target material increases. A current flowing in the X-ray tube 111 is referred to as “tube current” and is represented as mA (mean amperage). As tube current increases, the number of thermoelectrons emitted from the filament increases and as a result, a dose of the X-rays (energy of photons) generated by collision of thermoelectrons with the target material 111d increases.

Accordingly, energy of X-rays is controlled by tube voltage, and intensity or dose of X-rays is controlled by tube current and X-ray exposure time. More specifically, when the emitted X-rays have a predetermined energy band, the energy band is defined by an upper limit and a lower limit. The upper limit of the energy band, that is, a maximum energy of the emitted X-rays, is controlled by a level of tube voltage and the lower limit of the energy band, that is, a minimum energy of the emitted X-rays, is controlled by a filter. When low energy X-rays are filtered through a filter, a mean energy of emitted X-rays is increased.

The X-ray detector assembly 120 is also called a “bucky”, which may include the object contact area 127 contacting the object, an X-ray detector 121 to detect X-rays and an insulation member 123 to prevent transfer of heat to the X-ray detector 121. The object contact area 127 and the insulation member 123 will be described later and a configuration of the X-ray detector 120 will be described below in detail.

The X-ray detector 120 may detect X-rays having transmitted through the object and may convert the detected X-rays into an electric signal to obtain an image signal. The image signal may indicate an intensity of X-rays incident upon respective pixels. The image signal may be output to the controller 130. The controller 130 may produce an X-ray image of an object using the image signal.

Generally, the X-ray detector is classified according to constitutional component, conversion mode of detected X-rays into electric signals and acquisition mode of image signal.

First, according to constitutional component, the X-ray detector is divided into an X-ray detector including a single element and an X-ray detector including a hybrid element.

In the X-ray detector including a single element, a portion to detect X-rays and generate an electric signal, and a portion to read the electric signal and perform processing on the same are formed of a single material of semiconductor, or produced by a single process. For example, one light-receiving element, such as a charge coupled device (CCD) or complementary metal oxide semiconductor (CMOS), is used as the X-ray detector including a single element.

In the X-ray detector including a hybrid element, a portion to detect X-rays and generate an electric signal and a portion to read the electric signal and perform processing on the same are formed of different materials or are produced by different processes. For example, X-rays are detected using a light-receiving element such as a photodiode or CdZnTe and an electric signal is read and processed using a CMOS ROIC (read-out integrated circuit), or X-rays are detected using a strip detector and an electric signal is read and processed using the CMOS ROIC, or a-Si or a-Se flat panel system is used.

In addition, the X-ray detector is classified into a direct conversion-type and an indirect conversion type depending on conversion mode of X-rays into electric signals.

In the direct conversion type, when X-rays are emitted, electron-hole pairs are temporarily produced in a light-receiving element, electrons move to an anode and holes move to a cathode through an electric field applied to both ends of the light-receiving element. The X-ray detector converts these movements into electric signals. A material for the light-receiving element of the direct conversion type includes a-Se, CdZnTe, Hgl₂, Pbl₂ or the like.

In the indirect conversion type, a scintillator is provided between a light-receiving element and an X-ray source, which reacts with X-rays emitted from the X-ray source to emit photons having a visible range of wavelength, and the light-receiving element senses the photons and converts the same into an electric signal. A material for the light-receiving element of the indirect conversion-type X-ray detector is, for example, a-Si and the scintillator is a thin film-type GADOX scintillator, microcolumn, needle structured type CSI (T1) or the like.

In addition, depending on image signal acquisition mode, the X-ray detector is classified into charge integration mode in which charges are stored for a predetermined time and a signal is obtained therefrom, and photon counting mode in which, whenever a signal is generated by a single X-ray photon, the number of photons having a threshold energy or higher is counted.

Embodiments are not limited to constitutional component, conversion mode into electric signals and acquisition mode of image signal and any type of X-ray detector may be applied to the X-ray detector 121. Furthermore, embodiments are not limited to the afore-mentioned mode and other methods for detecting X-rays, converting the same into electric signals, and acquiring the image signal may be employed.

Hereinafter, as an example, an X-ray detector 121 using a direct conversion mode in which an electric signal is directly obtained from X-rays and a hybrid mode in which a light-receiving element to detect X-rays is combined with a readout circuit will be described.

Referring to FIG. 5B, the X-ray detector 121 includes a light-receiving element 121-1 to detect X-rays and convert the same into an electric signal and a readout circuit 121-2 to read the electric signal. The readout circuit 121-2 has a 2-dimensional pixel array including a plurality of pixel regions. As a material constituting the light-receiving element 121-1, a single crystal semiconductor material may be used in order to secure high resolution, rapid response time and high dynamic area at low energy and at low dose and examples of the single crystal semiconductor material includes Ge, CdTe, CdZnTe, GaAs and the like.

The light-receiving element 121-1 is formed as a PIN photodiode in which a p-type layer 121-1c including p-type semiconductors aligned in the form of a two-dimensional pixel array is bonded to a high-resistance n-type semiconductor substrate 121-1b and the readout circuit 121-2 using a CMOS process is bonded to the light-receiving element 121-1 in each pixel. The CMOS readout circuit 121-2 is bonded to the light-receiving element 121-1 in a flip-chip bonding manner. The bonding is carried out by forming a bump 121-3 such as solder (PbSn) or indium (In), reflowing and compressing while heating.

The afore-mentioned structure is provided only as an example of the X-ray detector 121 and embodiments are not limited thereto.

The compression paddle assembly 140 may include a compression paddle 141 to compress the breast 30 and a paddle driving unit 142 to drive the compression paddle 141. The compression paddle 141 may be made of a transparent material, to have no effect on transmission of X-rays, and the paddle driving unit 142 may include a gear, motor or the like to manually or automatically move the compression paddle 141.

The heat source unit 150 may include a heat source 151 to emit heat and a power supply 152 to supply power to the heat source. The heat source 151 may heat an area contacting the breast 30 and may enable the patient to feel warm when the breast 30 is compressed. The heat source 151 may transfer heat by radiation. Heat generated by the heat source 151 may be radiant heat. For example, the heat source 151 may be implemented by an infrared lamp to generate infrared rays having a predetermined wavelength band.

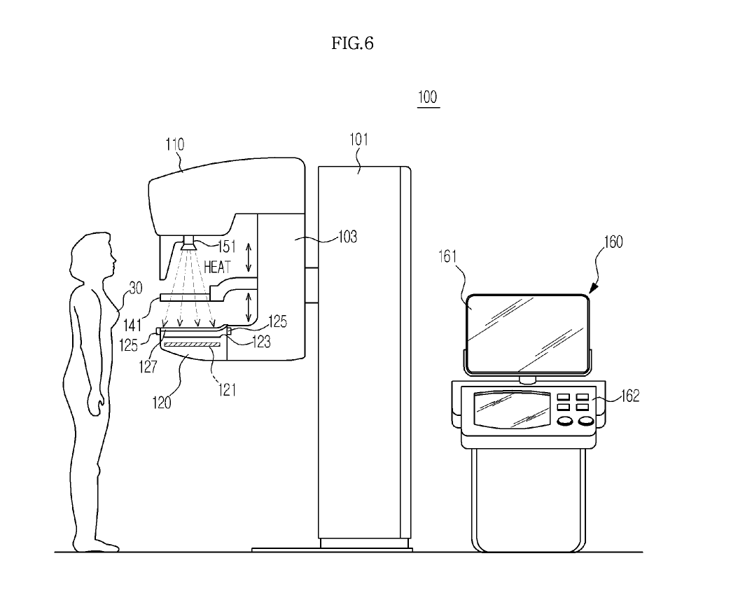

FIG. 6 is a view illustrating an external appearance of an X-ray imaging device according to one or more embodiments. A detailed operation of the X-ray imaging device 100 will be described with reference to FIG. 6 together with FIGS. 4 to 5B.

Referring to FIG. 6, the X-ray source 110, the X-ray detector module 120 and the compression paddle 141 may be connected to a frame 103 to support the same, and the frame 103 may be mounted on the gantry 101. As described above, X-rays may be generated by applying power to the X-ray tube 111. In this regard, a high-voltage generator may be provided in the gantry 101 to supply high voltage to the X-ray tube 111.

The compression paddle 141 may move in a vertical direction along the frame 103 and the paddle driving unit 142 to drive the compression paddle 141 may be mounted in the frame 103. The patient places the breast 30 on the object contact area 127 for X-ray imaging. The X-ray detector assembly 120 may also function to support the object 30. The object contact area 127 may be made of a material which reduces absorption of X-rays and may be for example made of carbon as a main component.

In addition, the compression paddle 141 may compress the breast 30 and may control a thickness of the breast 30 to be suitable for X-ray imaging. The compression paddle 141 may be manually controlled by a user or automatically controlled according to predetermined value. For example, when the thickness of the object 30 required for X-ray imaging is set to 5 cm, the controller 130 may transmit a control signal to the paddle driving unit 142 to move the compression paddle 141 to a position spaced by 5 cm from the object contact area 127.

During X-ray imaging, the area contacting the breast 30 may be a lower part of the object contact area 127 and the compression paddle 141. As described above, the X-ray detector 121 includes a semiconductor element. Because the X-ray detector 121 is sensitive to temperature, constitutional components including the X-ray detector assembly 120, of the X-ray imaging device 100 may be maintained at a low temperature.

The heat source 151 may be mounted at any position to heat the compression paddle 141 and the object contact area 127, and the power supply 152 may be mounted in the gantry 101. For example, as shown in FIG. 6, the heat source 151 may be mounted in a lower part of the X-ray source 110 and may emit heat from an upper part of the compression paddle 141 and the object contact area 127. The heat source 151 may be mounted at a position which does not shield the window such that it does not affect X-ray emission of the X-ray source 110.

Although the upper part of the compression paddle 141 and the object contact area 127 may be heated by the heat source 151, it may be necessary to maintain the temperature of the X-ray detector 121 at a predetermined level suitable for characteristics thereof. Accordingly, an insulation member 123 may be provided between the object contact area 127 and the X-ray detector 121 to block transfer of heat to the X-ray detector 121. The blocking of heat may be carried out by absorbing or reflecting heat.

For example, the insulation member 123 may be implemented as an insulating film coated with a heat-blocking material. The insulating film may be formed by coating a synthetic resin film for example a polyester (PET) film as a base film, with a material blocking infrared light with a wavelength range of about 0.75 μm to 1 mm.

Alternatively, a coating layer having an insulation property may be formed by directly coating an X-ray grid or the X-ray detector 121 with a heat-blocking material.

Meanwhile, the insulation member 123 transmits X-rays having transmitted through the object 30, instead of blocking the same, since it is disposed on a front surface of the X-ray detector 121. Accordingly, an infrared light-blocking material coated on the insulation member 123 may not block X-rays having a wavelength range of 0.001 nm to 10 nm. Furthermore, when X-ray blocking of the insulation member 123 is not negligible, the effect of the insulation member 123 may be offset through correction of the X-ray image, as described below.

The thickness of the insulation member 123 may be determined while taking into consideration X-ray transmittance and a space between the object contact area 127 and the X-ray detector 121 (or X-ray grid) in the X-ray detector assembly 120.

The controller 130 may control overall operations of the X-ray imaging device 100, and may receive X-ray data from the X-ray detector 121 to form an X-ray image, or may control generation and emission of X-rays by the X-ray source 110, or may control the compression paddle 141, or may control an amount of heat generated by the heat source 151.

The X-ray imaging device 100 may include a host device 160 as a main control device, and the host device 160 may also be called a workstation or console. The host device 160 may be a user interface, which may include a display unit 161 to display an X-ray image and other pictures associated with control of the X-ray imaging device 100 and an input unit 162 to receive a control instruction from the user.

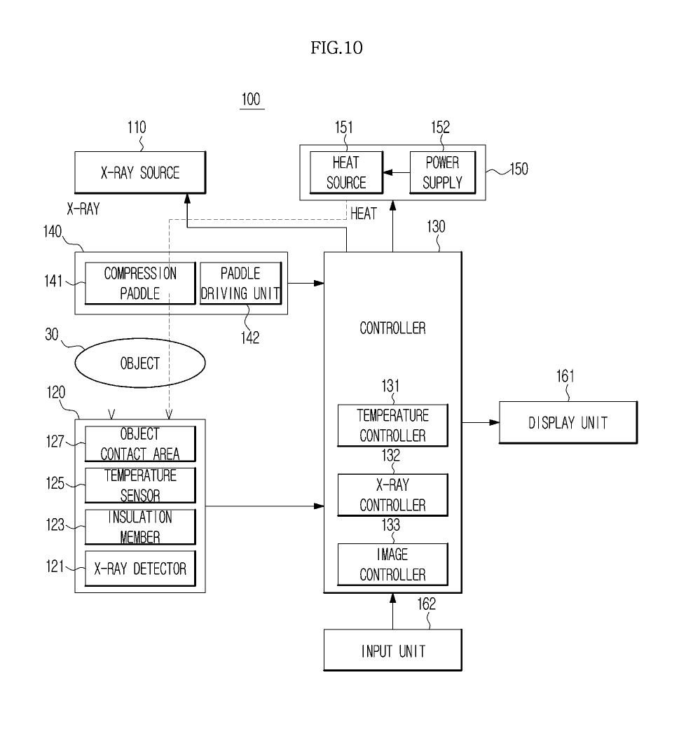

The controller 130 may be limited to one physical module. A module to perform one operation may be provided in the host device 160 and a module to perform another operation may be provided in the gantry 101 or the frame 103. A detailed operation of the controller 130 will be described later with reference to FIG. 10.

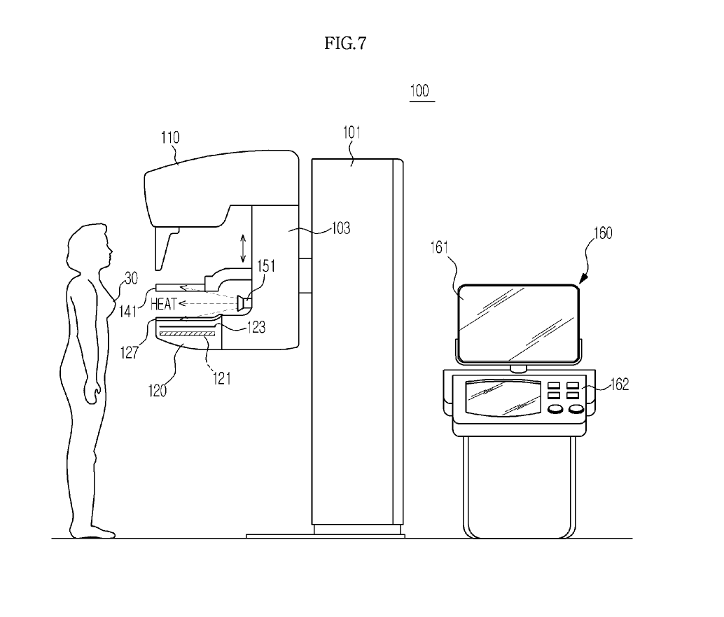

FIG. 7 is a view illustrating an external appearance of the X-ray imaging device according to one or more embodiments.

As described above with reference to FIG. 6, the heat source 151 may be mounted in a lower part of the X-ray source 110 to emit heat from an upper part of the compression paddle 141 and the object contact area 127. However, as described above, the heat source 151 may be mounted at any position enabling the lower part of the compression paddle 141 and the object contact area 127 to be heated. As shown in FIG. 7, the heat source 151 may be mounted in an area provided between the compression paddle 141 and the X-ray detector assembly 120 in the frame 103 to emit heat toward the front of the patient.

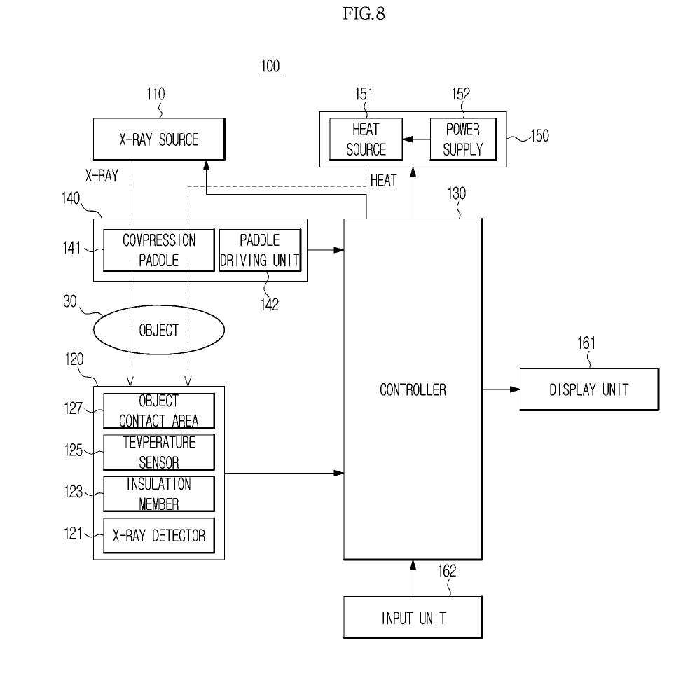

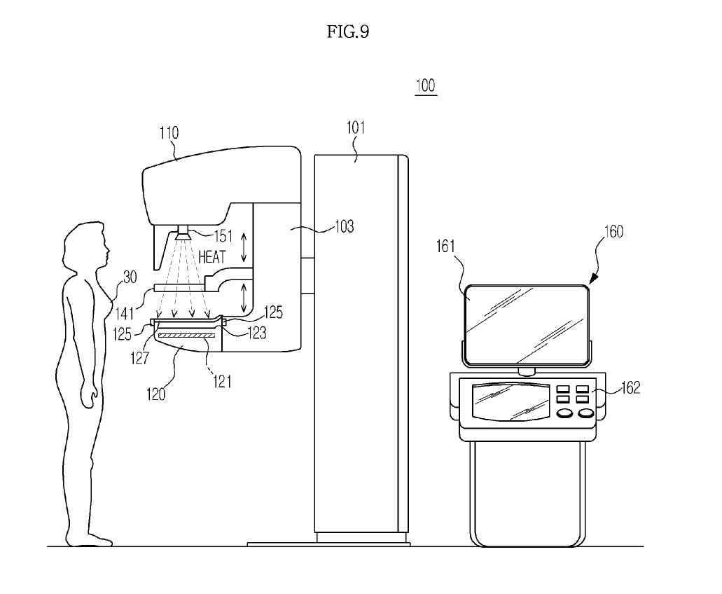

FIG. 8 is a block diagram illustrating a controlled configuration of an X-ray imaging device according to one or more embodiments further including a temperature sensor and FIG. 9 is a view illustrating an external appearance of an X-ray imaging device according to or more embodiments further including a temperature sensor.

The X-ray imaging device 100 may automatically control an amount of heat generated by the heat source 151. For this purpose, temperature information of an area in need of temperature control may be required. Accordingly, as shown in FIG. 8, the X-ray imaging device 100 may further include a temperature sensor 125 to sense a temperature of the object contact area 127 or an adjacent area.

As shown in FIG. 9, the temperature sensor 125 may be mounted on an end of the object contact area 127 or on both ends of the object contact area 127 so as to possibly improve accuracy of temperature sensing. In this case, the temperature sensor 125 may be mounted in a region not corresponding to the X-ray detector 121 so as to possibly not have an effect on X-ray detection of the X-ray detector 121. The region not corresponding to the X-ray detector 121 is an area which is not included in a passage of X-rays incident upon the X-ray detector 121. FIG. 9 illustrates only an example of a position at which the temperature sensor 125 is mounted, and the temperature sensor 125 may be mounted at any position which does not correspond to the X-ray detector 121 and enables sensing of the temperature of the object contact area 127 or an adjacent area.

In addition, the temperature sensor 125 may also be mounted on the compression paddle 141 to sense a temperature of the compression paddle 141, or it may be mounted only on the compression paddle 141. The temperature sensor 125 mounted on the compression paddle 141 may also be mounted in a region so as not to interfere with passage of X-rays.

FIG. 10 is a block diagram illustrating a detailed configuration of a controller of an X-ray imaging device according to one or more embodiments.

Referring to FIG. 10, the controller 130 may include a temperature controller 131 to control an amount of heat generated by the heat source unit 151 and thereby control a temperature of the area contacting the object 30, an X-ray controller 132 to control X-rays generated and emitted for X-ray imaging and an image controller 133 to form an X-ray image of the object 30 using X-rays detected by the X-ray detector 121.

The temperature controller 131 may turn on the heat source during a period in which X-ray imaging is not performed (hereinafter, referred to as rest period). Specifically, when the X-ray imaging device 100 is powered on, the temperature controller 131 may transmit a control signal to the heat generator 150 to heat an area contacting the object 30. The control signal transmitted to the heat generator 150 may be input to the power supply 152 and the power supply 152 may supply power to the heat source 151 to enable the heat source 151 to emit heat.

When X-ray imaging starts, the temperature controller 131 may transmit a control signal to the heat generator 150 to cut off power from the power supply 152 to the heat source 151. It may be determined that X-ray imaging starts when power is supplied to the X-ray source 110 or a user inputs a separate instruction through the input unit 162 for X-ray imaging.

The temperature controller 131 may maintain the temperature of the area contacting the object 30, that is, the compression paddle 141 and the object contact area 127 within a predetermined range. For this purpose, the temperature sensor 125 may sense a temperature in real-time or on a constant cycle and may transmit the temperature to the temperature controller 131, and the temperature controller 131 may control an amount of heat generated by the heat source 151, based on the sensed temperature.

The amount of heat generated by the heat source 151 may depend on a level of power supplied from the power supply 152. Accordingly, the temperature controller 131 may control the amount of heat generated by the heat source 151 by controlling a level of power supplied from the power supply 152 to the heat source 151.

Specifically, when the sensed temperature is lower than a predetermined lower limit, the temperature controller 131 may transmit a control signal to the heat generator 150 to increase an amount of heat generated by the heat source 151 and when the sensed temperature is higher than the predetermined upper limit, the temperature controller 131 may transmit a control signal to the heat generator 150 to decrease an amount of heat generated by the heat source 151, or may cut off power to the heat source 151 to stop heat generation for a predetermined time.

The predetermined lower and upper limits may fall within a temperature range to be maintained and may be correspond to lower and upper limits within the temperature range, or may be other values within the temperature range determined while taking consideration into a time taken for temperature variation caused by variation in generated heat amount.

The X-ray controller 132 may control imaging parameters such as levels of tube voltage and tube current supplied to the X-ray source 110 and X-ray exposure time and thereby controls energy and dose of X-rays generated by the X-ray source 110. The X-ray controller 132 may control X-rays according to user control instructions input by the input unit 162, and perform pre-shot before main imaging and thereby may perform auto-exposure control (AEC) suitable for characteristics of the object 30.

The image controller 133 may produce an X-ray image displayed on the display unit 161 using the image signal transmitted from the X-ray detector 121. The image signal transmitted from X-ray detector 121 may be a voltage signal or a signal corresponding to the number of photons, depending on image signal acquisition mode of the X-ray detector 121. The image signal may be transmitted to individual pixels and may become an X-ray image per se due to difference in brightness between pixels. However, the image controller 133 may perform image processing such as noise reduction or flat-field correction on the image signal to produce an X-ray image to be displayed through the display unit 161.

In addition, the image controller 133 may perform a correction operation so as to minimize effects of the insulation member 123. Since the insulation member 123 is disposed between the object contact area 127 and the X-ray detector 121, the insulation member 123 may have an effect on X-ray imaging when it is made of a material that absorbs X-rays. Accordingly, when the insulation member 123 has an effect on transmission of X-rays, the image controller 133 may perform a correction operation to offset this effect in individual pixels, while taking into consideration X-ray absorption properties.

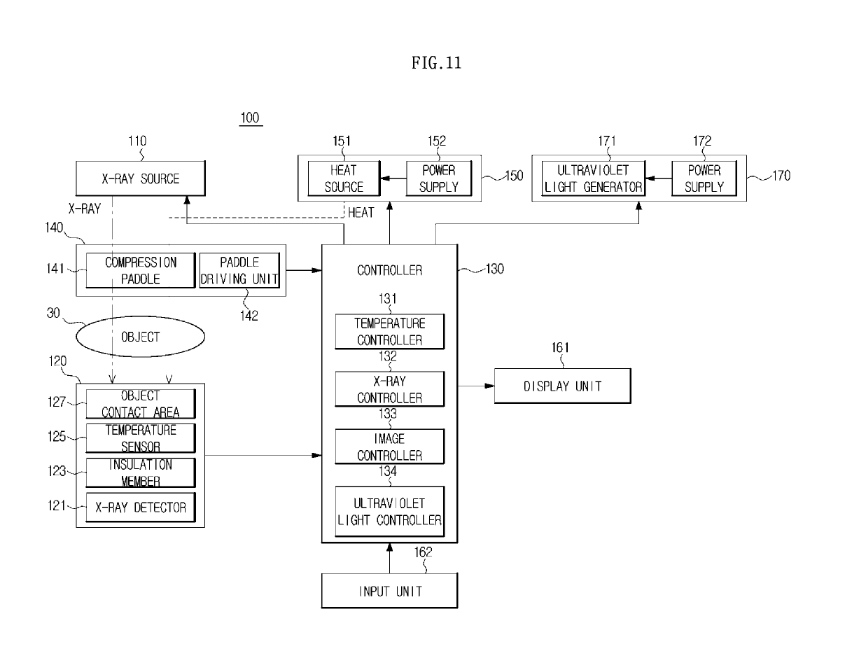

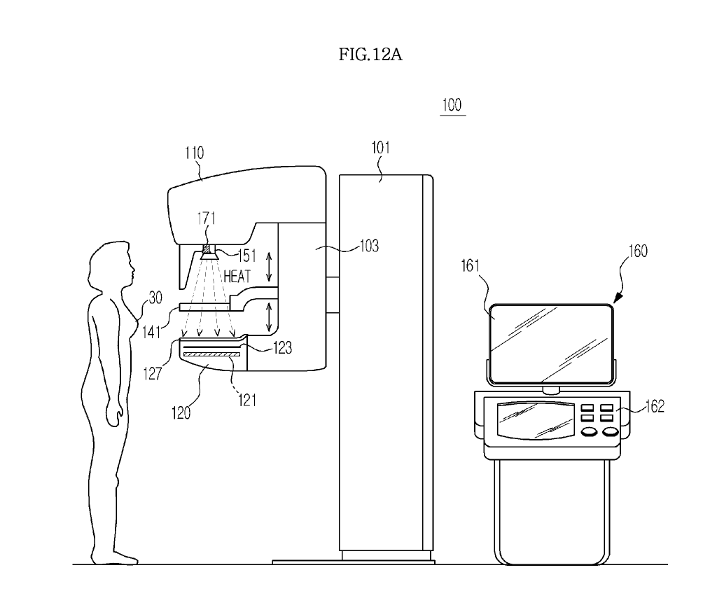

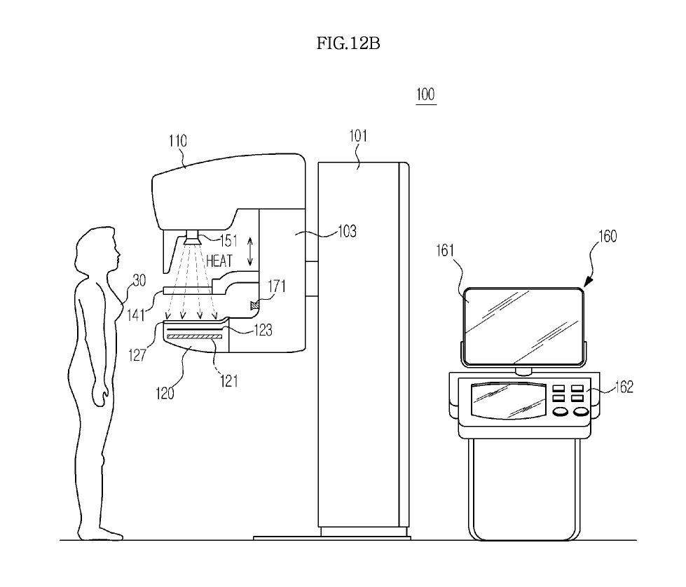

FIG. 11 is a block diagram illustrating a controlled configuration of an X-ray imaging device according to or more embodiments further including an ultraviolet light generator, and FIGS. 12A and 12B are views illustrating an external appearance of X-ray imaging device according to or more embodiments further including an ultraviolet light generator.

Referring to FIG. 11, the X-ray imaging device 100 may further include an ultraviolet light generation unit 170 to generate ultraviolet light and the ultraviolet light generation unit 170 may include an ultraviolet light generator 171 to generate ultraviolet light and a power supply 172 to supply power to the ultraviolet light generator 171.

In addition, the controller 130 may further include an ultraviolet light controller 134 to control the ultraviolet light generation unit 170.

For example, the ultraviolet light generator 171 may generate ultraviolet light having a wavelength of, for example, about 250 nm to 260 nm and thereby may sterilize the lower part of the compression paddle 141 and the object contact area 127. Ultraviolet sterilization may also be performed during a rest period. When X-ray imaging finishes, the ultraviolet light controller 134 may transmit a control signal to the ultraviolet light generation unit 170 to allow the power supply 172 to supply power to the ultraviolet light generator 171. The ultraviolet light generator 171 may be turned on upon receiving power. When X-ray imaging starts, the ultraviolet light controller 134 may transmit a control signal to the ultraviolet light generation unit 170 to cut off power from the power supply 172 to the ultraviolet light generator 171. When the power is cut off, the ultraviolet light generator 171 may be turned off.

As shown in FIG. 12A, the ultraviolet light generator 171 may be mounted in a lower part of the X-ray source 110 to emit ultraviolet light from an upper part of the compression paddle 141 and the object contact area 127.

Alternatively, as shown in FIG. 12B, the ultraviolet light generator 171 may be mounted in an area provided between the compression paddle 141 and the X-ray detector assembly 120 on the front surface of the frame 103 to emit ultraviolet light from a side of the compression paddle 141 and the object contact area 127.

FIGS. 12A and 12B illustrate an example of a position at which the ultraviolet light generator 171 is mounted However, the ultraviolet light generator 171 may be mounted at any position of the compression paddle 141 and the object contact area 127 to which ultraviolet light is emitted.

The X-ray imaging device 100 may be used for a plurality of patients. Accordingly, microorganisms such as bacteria may be readily propagated on the compression paddle 141 and the object contact area 127 contacting the patients. The microorganisms may be destroyed and sterilization effects may be thus obtained when ultraviolet light is emitted to the compression paddle 141 and the object contact area 127.

Hereinafter, an embodiment of an X-ray imaging device to automatically sterilize the object contact area using ultraviolet light will be described in more detail.

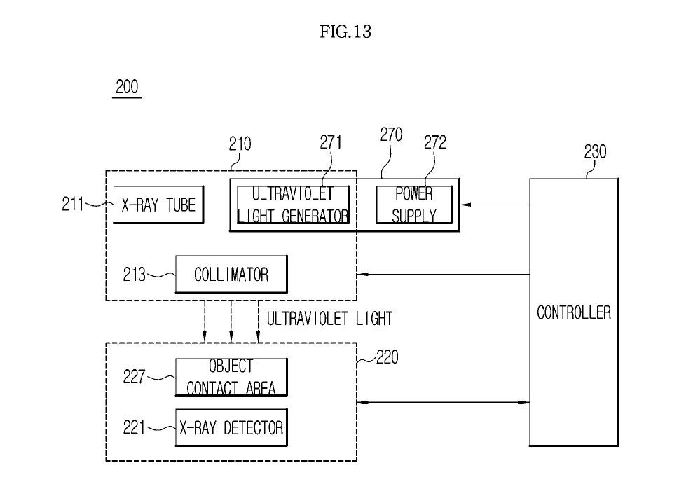

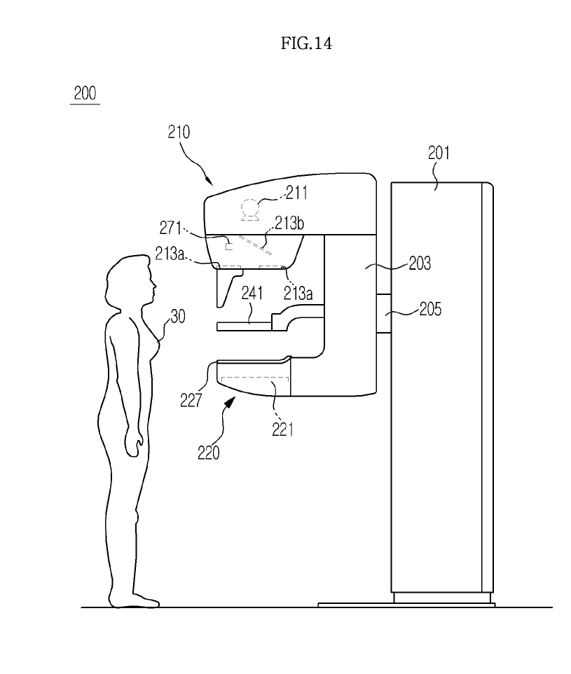

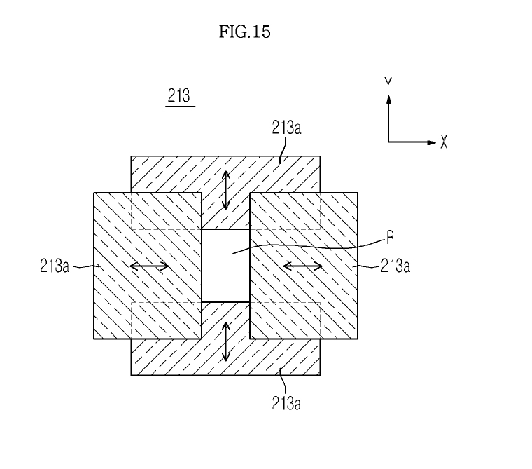

FIG. 13 is a block diagram illustrating an X-ray imaging device according to one or more embodiments. FIG. 14 is a view illustrating an outer appearance of an X-ray imaging device according to one or more embodiments. FIG. 15 is a view illustrating an example of a collimator included in an X-ray imaging device according to one or more embodiments.

The X-ray imaging device 200 may include an X-ray source 210 that may include an X-ray tube 211 to generate and emit X-rays and a collimator 213 to control an emission area of X-rays, an X-ray detector assembly 220 that may include an X-ray detector 211 to detect X-rays transmitting through the object and an object contact area 227 that may be mounted on the X-ray detector 211 and that may contact the object, a ultraviolet light generation unit 270 to generate ultraviolet light and thereby possibly sterilize the object contact area 227 and a controller 230 to control the ultraviolet light generation unit 270 to generate ultraviolet light during an idle period in which X-ray imaging is not performed.

Referring to FIG. 14, the X-ray source 210, the X-ray detector assembly 220 and a compression paddle 241 may be connected to a frame 203 supporting the same and the frame 203 may be mounted on a gantry 201.

Detailed description of the X-ray tube 211 is the same as that of the X-ray tube 111 according to the embodiment described above. Accordingly, a power supply to generate high voltage may be provided inside or outside of the gantry 201 to supply the high voltage to the X-ray tube 211.

The compression paddle 241 may move in a vertical direction along the frame 203 and the paddle driving unit (not shown) to drive the compression paddle 241 may be mounted in the frame 203. In one example, a patient may place the breast 30 on the object contact area 227 for X-ray imaging. The X-ray detector assembly 220 may also support the object 30. The object contact area 227 may be made of a material which may reduce absorption of X-rays and may, for example, be made of carbon as a main component.

The collimator 213 may be mounted under the X-ray tube 211 to control an emission area of X-rays emitted from the X-ray tube 211. The collimator 213 may be implemented, for example, in an integral form with the X-ray source 210, as shown in FIG. 14, or may be implemented in a separate module from the X-ray source 210.

FIG. 15 is a view showing an example of the collimator 213 seen from above. The collimator 213 may include at least one shutter 213a to shield X-rays. The shutter 213a may be also referred to as a blade. Referring to FIG. 15, the collimator 213 may include a plurality of shutters 213a to vary emission areas of X-rays and the shutters 213a may be each independently moved in an X- or Y-axis direction. Although not shown in the drawings, the collimator 213 may further include a shutter driving unit to move the shutters 213a and the shutter driving unit may include an actuator such as a motor.

The shutters 213a may be formed of a material having a high bandgap, thus absorbing X-rays to shield the X-rays and X-rays may be emitted to the object 30 through an opening R formed by the shutters 213a. In one or more embodiments, the opening R is referred to as an X-ray transmittance area.

Referring to FIGS. 13 and 14 again, the ultraviolet light generation unit 270 may include an ultraviolet light generator 271 to generate ultraviolet light and a power supply 272 to supply power to the ultraviolet light generator 271. The ultraviolet light generator 271 may be implemented with a light source to generate light of a ultra-violet band. For example, the ultraviolet light generator 271 may generate light having a wavelength of about 100 to 380 nm, more specifically, of about 250 to 260 nm. The light having the wavelength range defined above exhibits superior sterilization effect.

The power supply 272 may be provided inside of the gantry 201 and may be used in conjunction with the power supply to supply power to the X-ray tube 211, but the embodiment of the X-ray imaging device 200 is not limited thereto and there is no particular limitation as to position of the power supply 272 and whether or not the power supply supplies power to other components.

The ultraviolet light generator 271 may be mounted inside of the collimator 213 and areas in which ultraviolet light is emitted from the ultraviolet light generator 271 may be controlled by the shutters 213a of the collimator 213. The ultraviolet light may have negative effects on human body when they are directly emitted thereto and may cause yellowing when they are directly emitted to articles. Accordingly, the collimator 213 limits emission areas of ultraviolet light to the object contact area 227 requiring sterilization, thereby possibly preventing negative effects such as unnecessary exposure of ultraviolet light to a user such as radiological technologist or doctor, or a patient, and yellowing of surrounding components. As such, emission areas of ultraviolet light may be limited without an additional component by mounting the ultraviolet light generator 271 inside of the collimator 213.

Ultraviolet light may be directly emitted to the shutters 213a according to position of the mounted ultraviolet light generator 271. As shown in FIG. 14, when the ultraviolet light generator 271 is mounted in a position in which ultraviolet light is not directly emitted to the shutters 213a, a reflection plate 213b provided in the collimator 213 may be used. Hereinafter, a control operation of emission areas of ultraviolet light by the collimator 213 will be described in detail.

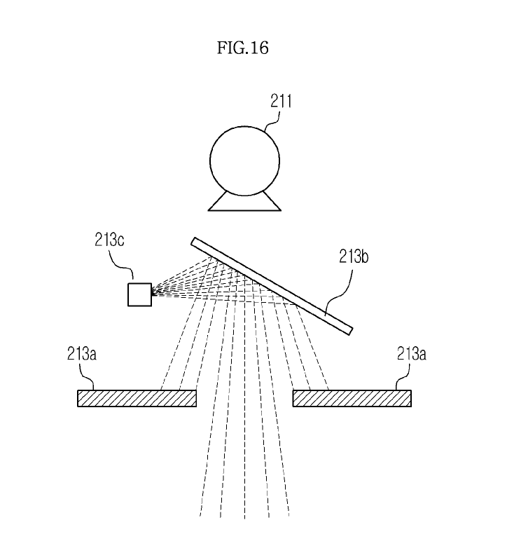

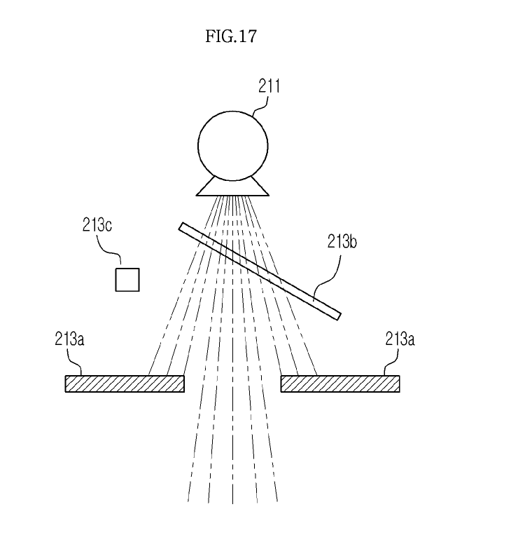

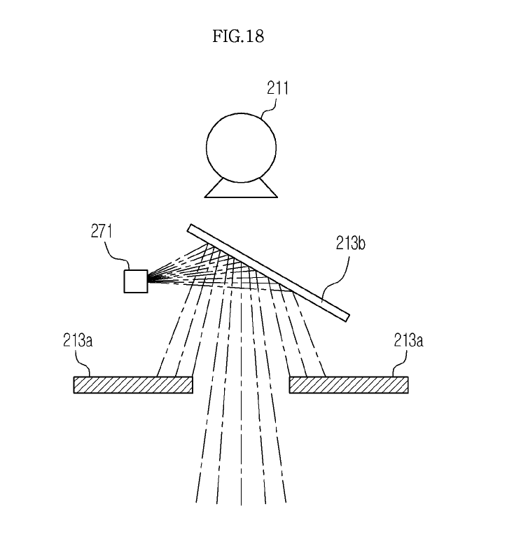

FIG. 16 is a view illustrating an indication operation of an X-ray imaging area by a visible light source provided in a collimator according to one or more embodiments. FIG. 17 is a view illustrating a control operation of emission areas of X-rays emitted from an X-ray tube, by a shutter according to one or more embodiments. FIG. 18 is a view illustrating a control operation of emission areas of ultraviolet light emitted from an ultraviolet light generator, by a shutter according to one or more embodiments. Only components related to the corresponding operations are shown in FIGS. 16 to 18.

Referring to FIG. 16, the collimator 213 may be provided with a visible light source 213c to indicate an X-ray imaging area, that is, an emission area of X-rays, before X-ray imaging.

Visible light emitted from the visible light source 213c may be reflected by the reflection plate 213b mounted to be inclined to an emission direction of visible light and may be emitted toward the shutters 213a. The shutters 213a may absorb both X-rays and visible light. For this reason, only visible light incident upon the opening R may reach the object contact area 227. Accordingly, the user may confirm visible light reaching the object contact area 227 by the naked eye and thereby may previously determine the X-ray imaging area.

The user may start X-ray imaging when the X-ray imaging area indicated by visible light is considered to be reasonable. As shown in FIG. 17, X-rays generated and emitted by the X-ray tube 211 may be emitted to the breast 30 while an emission area of the X-rays is controlled by the shutters 213a of the collimator 213. When the reflection plate 213b is formed of a material transmitting X-rays, the reflection plate 213b may be disposed in an X-ray emission route, as shown in FIG. 16, but the position of the reflection plate 213b is not limited to that illustrated in FIG. 16 and may be mounted out of the X-ray emission route.

Meanwhile, the user may change the X-ray imaging area by moving the shutters 213a using the shutter driving unit, when the X-ray imaging area indicated by visible light is determined to be reasonable.

Referring to FIG. 18, the ultraviolet light generator 271 may be disposed parallel to the visible light source 213c. FIG. 18 is a view illustrating components inside the X-ray source 210 seen from a side and thus shows only the ultraviolet light generator 271, assuming that one of the ultraviolet light generator 271 and the visible light source 213c is seen.

The reflection plate 213b may be formed of a material which reflects both visible light and ultraviolet light. Accordingly, after the ultraviolet light generator 271 emits ultraviolet light to the reflection plate 213b, the emitted ultraviolet light may be reflected by the reflection plate 213b and may travel toward the shutter 213a. The shutter 213a may absorb both X-rays and ultraviolet light and, thus, only ultraviolet light incident upon the opening R may reach the object contact area 227.

Accordingly, the controller 230 may control such that the shutter driving unit moves the shutter 213a to a position corresponding to a predetermined ultraviolet light emission area. Here, the position corresponding to the predetermined ultraviolet light emission area may mean a position of the shutter 213a which enables position and area of the opening R formed by the shutters 213a to be equivalent to or correspond to position and area of the predetermined ultraviolet light emission area.

The controller 230 may control the position of the shutters 213a such that ultraviolet light is emitted only to the object contact area 271, thereby possibly reducing unnecessary exposure of ultraviolet light to humans or surrounding components.

Meanwhile, as a specific example of a region corresponding to the object contact area 271, the region corresponding to position and area of the object contact area 271, may be set as a basis of the ultraviolet light emission area and may be changed by the user or the controller 230, as necessary.

As described above, ultraviolet light sterilization may be performed during an idle period in which X-ray imaging is not performed, because ultraviolet light is harmful to humans and affects X-ray imaging. Accordingly, the controller 230 may automatically determine the idle period in which X-ray imaging is not performed and may perform ultraviolet light sterilization. One or more detailed embodiments associated with these operations will be described below.

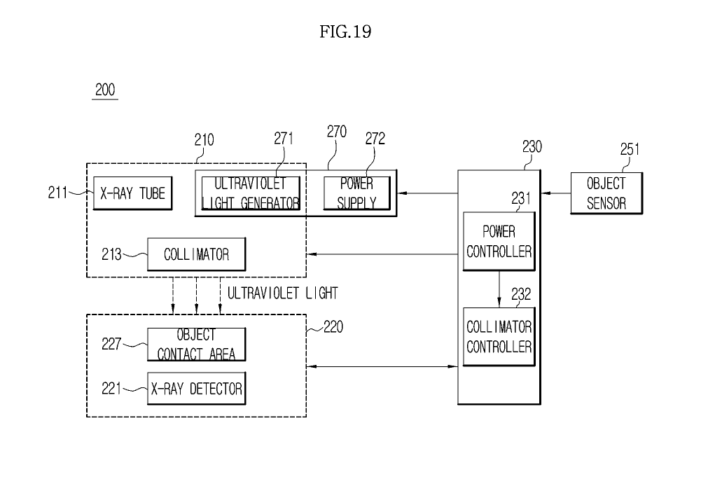

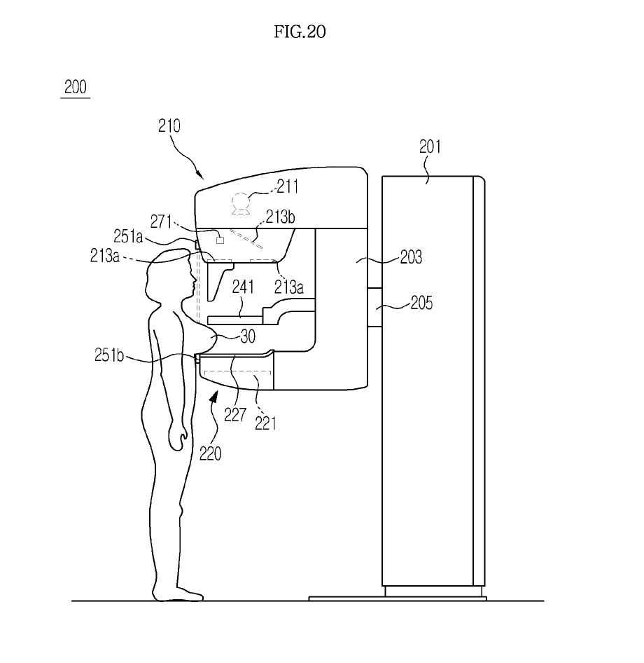

FIG. 19 is a block diagram illustrating an X-ray imaging device according to one or more embodiments further including an object sensor, and FIG. 20 is a view illustrating an outer appearance of an X-ray imaging device according to one or more embodiments further including an object sensor.

Referring to FIG. 19, the X-ray imaging device 200 may further include an object sensor 251 to sense an object, and the controller 230 may include a power controller 231 to control power applied to the ultraviolet light generator 271 and a collimator controller 232 to control position of the collimator 213, more specifically, position of the shutter 213a of the collimator 213.

The object sensor 251 may be implemented with a sensor to sense objects, for example, an optical sensor including an infrared sensor, a laser sensor, a visible sensor and the like, an ultrasonic sensor or a touch sensor. When the object sensor 251 is implemented with an infrared sensor, an ultrasonic sensor or a laser sensor, the sensor 251 may be implemented with a reflection-type sensor in which a light emitting unit to emit infrared light, ultrasonic waves or laser, and a light receiving unit to detect the same are disposed in the same side based on the object, or a transmission-type sensor in which the light emitting unit and the light receiving unit are disposed in different sides, but the embodiment of the X-ray imaging device 200 is not limited thereto and any sensor, for example, proximity sensor may be used as the object sensor 251 so long as it senses objects.

The operation of the controller 230 described with reference to FIG. 18 corresponds to an operation of the collimator controller 232. The power controller 231 may control power applied to the ultraviolet light generator 271, based on sensed results of the object sensor 251. Hereinafter, sensing of the object by the object sensor 251 and control of power applied to the ultraviolet light generator 271 by the power controller 231, based on sensing result of the object sensor 251 will be described in detail with reference to FIG. 20.

A transmission-type infrared sensor may be used as the object sensor 251 in one or more embodiments as shown in FIG. 20. Accordingly, the object sensor 251 may include a light emitting unit 251a and a light emitting unit 251b. As shown in FIG. 20, the light emitting unit 251a may be mounted in a portion of the X-ray source 210 and the light emitting unit 251b may be mounted in a portion of the X-ray detector assembly 220. Positions of the light emitting unit 251a and the light emitting unit 251b may be interchangeable.

In addition, position of the object to be sensed may be changed according to mount positions of the light emitting unit 251a and the light emitting unit 251b or emission direction of infrared light. As shown in FIG. 20, when the light emitting unit 251a is mounted in a position enabling the light emitting unit to emit infrared light downwardly in a vertical direction, the object disposed on the object contact area 227 may be sensed.

Alternatively, when the light emitting unit 251a is mounted in a position enabling the light emitting unit 251a to emit infrared light toward a patient, the object sensor 251 may sense the object 30 or patient adjacent to the object contact area 227 although the object may not be disposed on the object contact area 227.

An output signal of the object sensor 251 may be transmitted to the power controller 231 and the power controller 231 may control the power supply 272 based on the output signal of the light emitting unit 251b. As shown in FIG. 20, when the object 30 is disposed on the object contact area 227, only a part of infrared light emitted from the light emitting unit 251a may be emitted to the light emitting unit 251b and infrared light emitted from the light emitting unit 251a may not be emitted to the light emitting unit 251b. Accordingly, the power controller 231 may determine whether or not the object is disposed on the object contact area 227 using the output signal of the light emitting unit 251b indicating an amount of incident infrared light.

The power controller 231 may perform a control operation such that the ultraviolet light generator 271 is turned on, when the output signal of the object sensor 251 indicates absence of the object 30. Here, the absence of the object 30 means that the object 30 may not be disposed on the object contact area 227 or the object 30 may not be adjacent to the object contact area 227.

For example, the power controller 231 may determine that the object 30 is not present on the object contact area 227 when the output signal of the object sensor 251 is equivalent to or higher than a predetermined reference value. When the object 30 is determined to be absent, the power controller 231 may determine that a current stage is an idle period in which X-ray imaging is not performed and may perform a control operation such that the ultraviolet light generator 271 is turned on. That is, the power controller 231 may perform a control operation such that the power supply 272 may supply power to the ultraviolet light generator 271.

Meanwhile, the sensing operation of the object sensor 251 may be performed every predetermined time or every predetermined cycle or may continue for a predetermined time.

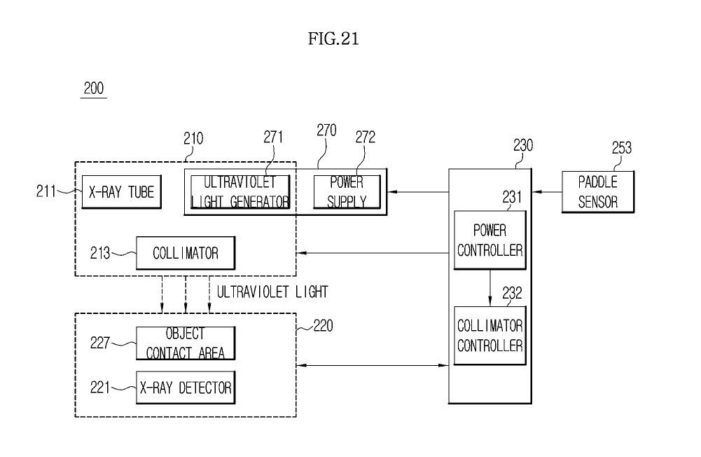

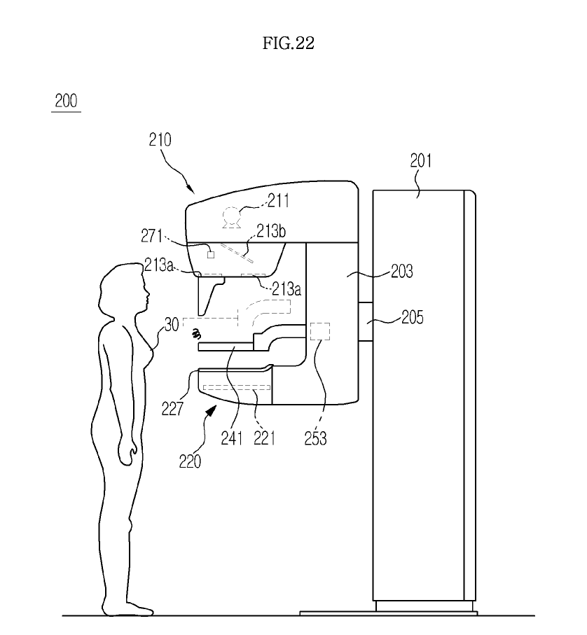

FIG. 21 is a block diagram illustrating a configuration of an X-ray imaging device according to one or more embodiments further including a paddle sensor, and FIG. 22 is a view illustrating an outer appearance of the configuration of an X-ray imaging device according to one or more embodiments further including a paddle sensor.

Referring to FIG. 21, the X-ray imaging device 200 may further include a paddle sensor 253 to sense a compression paddle 241 mounted in the frame 203 and the controller 230 may include a power controller 231 to control power applied to the ultraviolet light generator 271, and a collimator controller 232 to control position of a collimator 213, more specifically, position of a shutter 213a of the collimator 213.

The paddle sensor 253 may be implemented with a micro-switch, a limit switch or the like, which may sense the compression paddle 241 by mechanical contact or with a non-contact proximity sensor such as, for example, infrared sensor, ultrasonic sensor, laser sensor, or magnetic sensor, or the like. There is no limitation as to type of the paddle sensor 253 so long as the paddle sensor 253 senses the compression paddle 241 mounted in the frame 203.

The operation of the controller 230 described with reference to FIG. 18 corresponds to an operation of the collimator controller 232. The power controller 231 may control power applied to the ultraviolet light generator 271, based on sensing results of the paddle sensor 253. Hereinafter, sensing of the object by the object sensor 251 and control of power applied to the ultraviolet light generator 271 by the power controller 231, based on sensing results of the paddle sensor 253 will be described in detail with reference to FIG. 22.

Referring to FIG. 22, the compression paddle 241 may be removably mounted on the frame 203. In addition, a variety of the compression paddles 241 may be provided according to size or shape and the compression paddle 241 suited for conditions of X-ray imaging or the object 30 may be selected and mounted in the frame 203.

The paddle sensor 253 may be accommodated in the frame 203 and may sense the compression paddle 241 mounted in the frame 203. There is no limitation as to the position of the paddle sensor 253 so long as the paddle sensor 253 senses the compression paddle 241. The paddle sensor 253 may not necessarily be accommodated in the frame 203.

The output signal of the paddle sensor 253 may be transmitted to the power controller 231 and the power controller 231 may control the ultraviolet light generation unit 270 based on the output signal of the paddle sensor 253. The power controller 231 may perform a control operation such that the power supply 272 applies power to the ultraviolet light generator 271 to turn on the ultraviolet light generator 271 when the paddle sensor 253 indicates absence of the compression paddle 241. Here, the absence of the compression paddle 241 means that the compression paddle 241 may not be mounted in the frame 203.

When ultraviolet light is emitted in the absence of the compression paddle 241, ultraviolet light sterilization may be performed during an idle period in which X-ray imaging is not performed, and a phenomenon in which ultraviolet light does not reach the object contact area 227, in the case in which the compression paddle 241 disposed between the ultraviolet light generator 271 and the object contact area 227 is formed of a material not allowing permeation of ultraviolet light, may be prevented.

Meanwhile, the power controller 231 may perform a control operation such that the ultraviolet light generator 271 may be turned on in the case of the absence of the compression paddle 241 for a predetermined time or longer. Here, the predetermined time may distinguish the absence of the compression paddle 241 indicating the idle period from a temporary absence for replacement of the compression paddle 241 and a value of the predetermined time may be previously set and may be changed by a user.

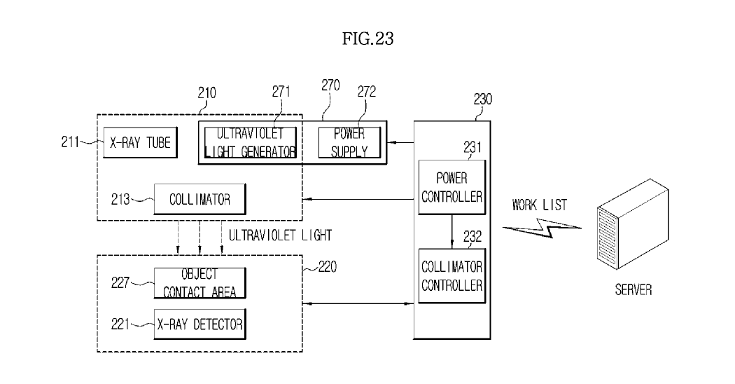

FIG. 23 is a block diagram illustrating a receiving operation of a work list from a server, in an X-ray imaging device according to one or more embodiments.

A patient explains his symptoms or shows a doctor a diseased area while taking a medical treatment and the doctor determines an imaging site and makes an imaging order according to conditions of the patient. The imaging order of the doctor may be transmitted to a server of a hospital through a network, the server may transmit the imaging order of the doctor to the X-ray imaging device 200 through the network and X-ray imaging may be performed according to the imaging order of the doctor. The imaging order transmitted from the server is referred to as a “work list”.

The power controller 231 may automatically determine the idle period based on the work list and may control the ultraviolet light generation unit 270. Specifically, the power controller 231 may analyze the work list, may determine whether or not remaining X-ray imaging to be further performed is present, and may perform a control operation such that the power supply 272 may apply power to the ultraviolet light generator 271 to turn on the ultraviolet light generator 271 when the remaining X-ray imaging is not present.

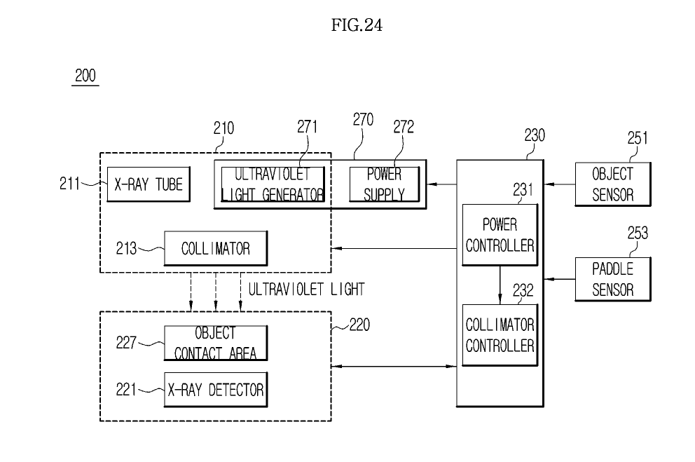

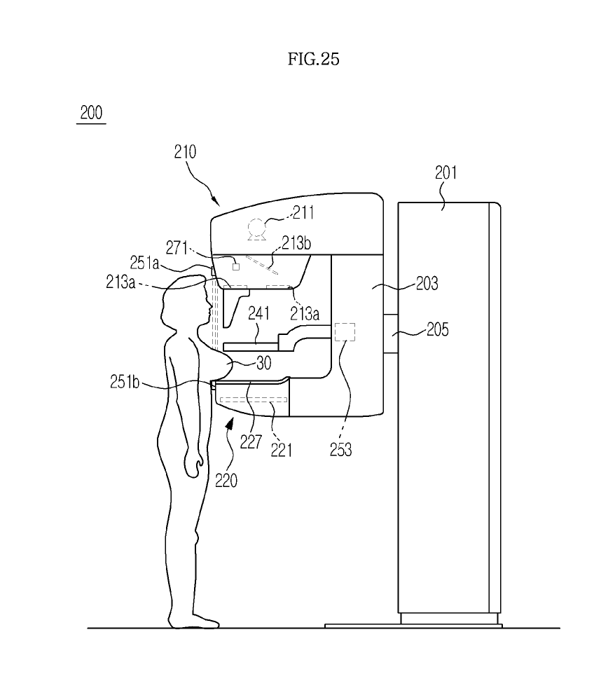

FIG. 24 is a block diagram illustrating a configuration of an X-ray imaging device according to one or more embodiments further including an object sensor and a paddle sensor, and FIG. 25 is a view illustrating an outer appearance of an X-ray imaging device according to one or more embodiments further including an object sensor and a paddle sensor.

Referring to FIGS. 24 and 25, the X-ray imaging device 200 may include both the object sensor 251 and the paddle sensor 253. Description of the object sensor 251 and the paddle sensor 253 is the same as described in FIGS. 19 to 22.

The power controller 231 may control the ultraviolet light generation unit 270 taking into consideration both output signal of the object sensor 251 and the output signal of the paddle sensor 253. Specifically, the power controller 231 may perform a control operation such that the power supply 272 may supply power to the ultraviolet light generator 271 to turn on the ultraviolet light generator 271, when the output signal of the object sensor 251 indicates absence of the object 30 and the output signal of the paddle sensor 253 indicates absence of the compression paddle 241.

As such, when the power controller 231 controls the ultraviolet light generation unit 270 while taking into consideration both the output signal of the object sensor 251 and the output signal of the paddle sensor 253, unnecessary generation of ultraviolet light may be prevented in the case in which the object 30 is not present, but the compression paddle 241 is present, and exposure of the object 30 to ultraviolet light may be prevented, in the case in which the compression paddle 241 is not present, but the object 30 is present.

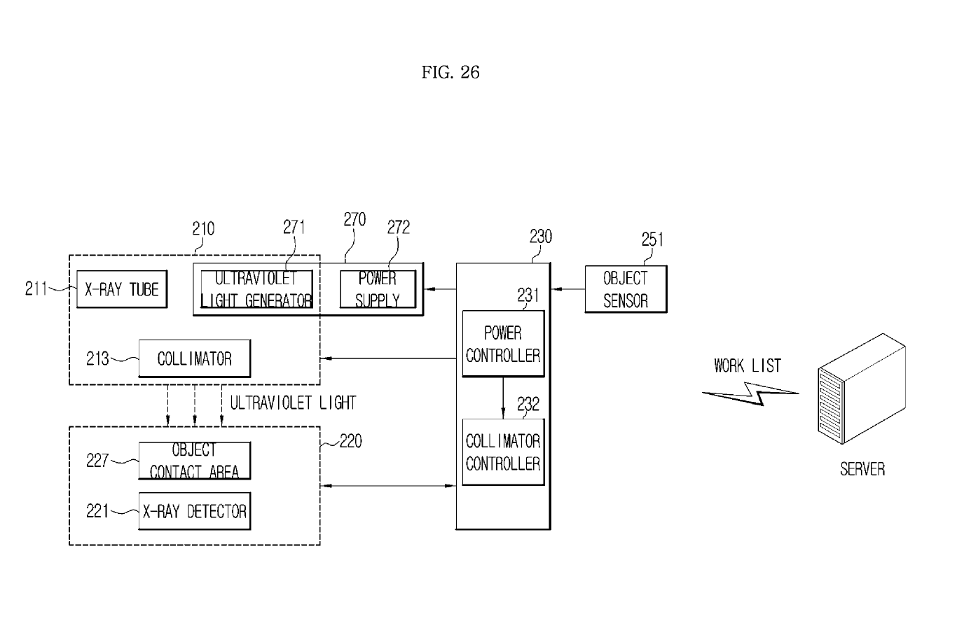

FIG. 26 is a block diagram illustrating a control operation of generation of ultraviolet light based on an output signal of an object sensor and a work list, in an X-ray imaging device according to one or more embodiments.

Referring to FIG. 26, the power controller 231 may perform a control operation such that the ultraviolet light generator 271 is turned on, based on output signal of the object sensor 251 and the work list. Specifically, the power controller 231 may perform a control operation such that the ultraviolet light generator 271 may be turned on in the case in which the output signal of the object sensor 251 indicates absence of the object 30 and remaining X-ray imaging is not present in the work list.

As such, the power controller 231 may control the ultraviolet light generation unit 270 in consideration of the output signal of the object sensor 251 and the work list, thereby possibly improving reliability of determination associated with the idle period.

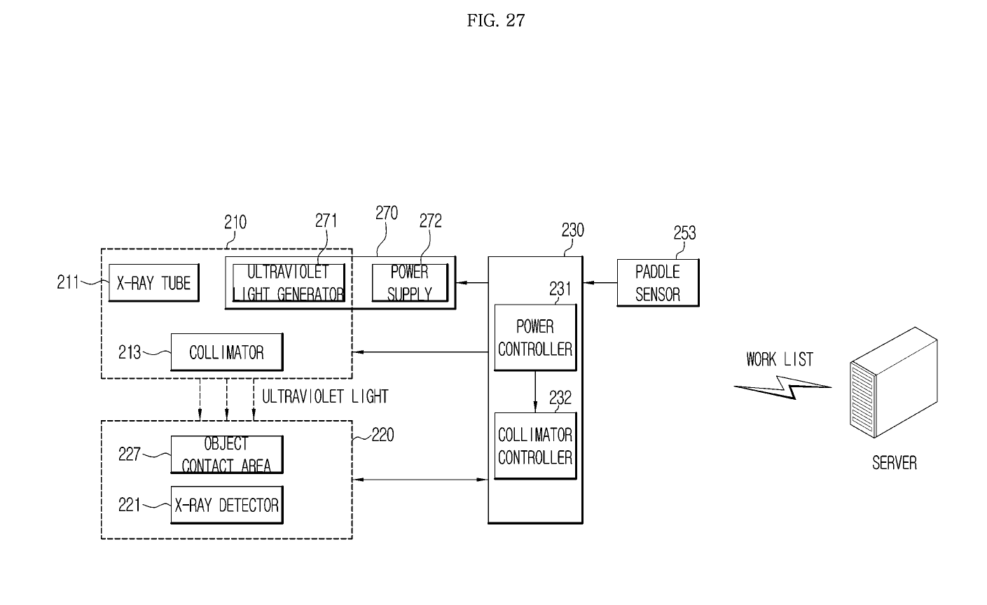

FIG. 27 is a block diagram illustrating a control operation of generation of ultraviolet light, based on an output signal of a paddle sensor and a work list, in an X-ray imaging device according to one or more embodiments.

Referring to FIG. 27, the power controller 231 may perform a control operation such that the ultraviolet light generator 271 is turned on, based on output signal of the paddle sensor 253 and the work list. Specifically, the power controller 231 may perform a control operation such that the ultraviolet light generator 271 may be turned on in the case in which the output signal of the paddle sensor 253 indicates absence of the compression paddle 241 and absence of remaining X-ray imaging in the work list.

Similarly, the power controller 231 may control the ultraviolet light generation unit 270 in consideration of the output signal of the paddle sensor 253 and the work list, thereby possibly improving reliability of determination associated with the idle period.

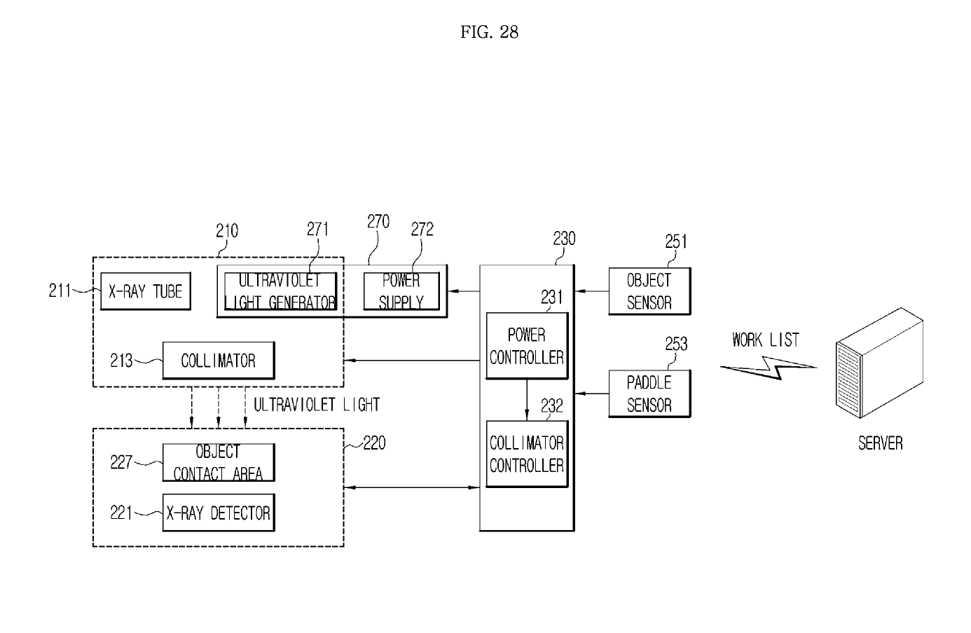

FIG. 28 is a block diagram illustrating a control operation of generation of ultraviolet light, based on an output signal of an object sensor, the output signal of a paddle sensor and a work list, in an X-ray imaging device according to one or more embodiments.

Referring to FIG. 28, the power controller 231 may perform a control operation such that the ultraviolet light generator 271 may be turned on, based on the output signal of the object sensor 251, the output signal of the paddle sensor 253 and the work list. Specifically, the power controller 231 may perform a control operation such that the ultraviolet light generator 271 may be turned on in the case in which the output signal of the object sensor 251 indicates absence of the object 30, the output signal of the paddle sensor 253 indicates absence of the compression paddle 241 and remaining X-ray imaging is not present in the work list.

As such, the power controller 231 may control the ultraviolet light generation unit 270 in consideration of all of the output signal of the object sensor 251, the output signal of the paddle sensor 253 and the work list, thereby possibly preventing exposure of the object 30 to ultraviolet light, unnecessary generation of ultraviolet light and possibly improving reliability of determination associated with the idle period.

The collimator controller 232 may control position of the shutter 213a of the collimator 213 to regulate emission areas of ultraviolet light when the power controller 231 determines a current stage to be an idle period according to the afore-mentioned embodiments. As described above, the position of the shutter 213a may be previously set or be changed by a user.

The power controller 231 may turn on the ultraviolet light generator 271 when the shutter 213a is disposed in a suitable position, and may automatically turn off the ultraviolet light generator 271 after a predetermined time. Here, the predetermined time refers to a time that may be required for ultraviolet light sterilization and may be previously set or be changed by the user.

Alternatively, the power controller 231 may automatically turn off the ultraviolet light generator 271 when the output signal of the object sensor 251 indicates presence of the object 30 or the output signal of the paddle sensor 253 indicates presence of the compression paddle 241

In addition, the power controller 231 may automatically turn off the ultraviolet light generator 271 upon receiving a new work list for X-ray imaging.

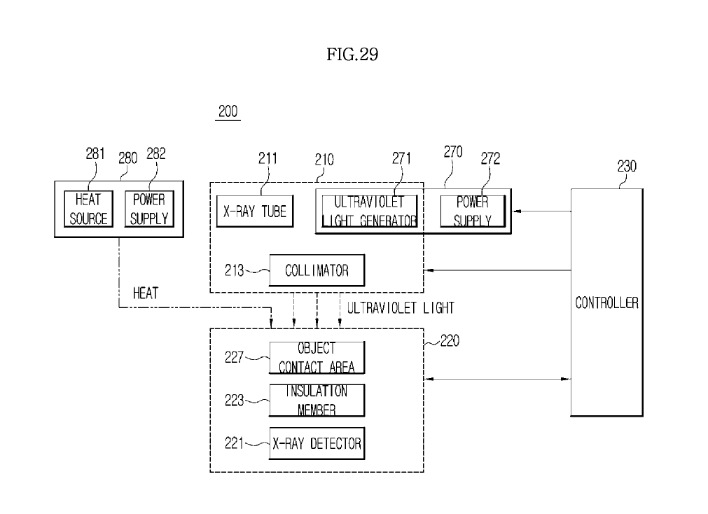

FIG. 29 is a block diagram illustrating a configuration of an X-ray imaging device according to one or more embodiments further including a heat source unit.

Referring to FIG. 29, the X-ray imaging device 200 may further include a heat source unit 280 to heat an area contacting the object 30 and the heat source unit 280 that may be included in the X-ray imaging device 200 may be the same as the heat source unit 150 described with reference to FIGS. 1 to 12B. In addition, the X-ray detector assembly 220 may further include an insulation member 223 to block heat transfer to the X-ray detector 211 and the insulation member 223 may be also the same as the insulation member 123 described with reference to FIGS. 1 to 12B.

The power controller 231 may control both the power supply 272 of the ultraviolet light generation unit 270 and the power supply 282 of the heat source unit 280, and the power supply 272 of the ultraviolet light generation unit 270 and the power supply 282 of the heat source unit 280 may be implemented with one power source.

Meanwhile, the X-ray imaging device 200 may further include at least one of the object sensor 251 and the paddle sensor 253, or receive a work list for the server. In addition, the X-ray imaging device 200 may turn on the ultraviolet light generator 271 or the heat source 281 according to presence of the object or the compression paddle 241 or presence of remaining X-ray imaging.

The power controller 231 may perform a control operation such that both the ultraviolet light generator 271 and the heat source 281 may be turned on during the idle period, with a proviso that the ultraviolet light generator 271 and the heat source 281 may not be simultaneously turned on. For example, when the X-ray imaging device 200 further includes the paddle sensor 253 and the object sensor 251, as shown in the embodiment of FIG. 24, the ultraviolet light generator 271 may be turned on, in the case in which the output signal of the object sensor 251 indicates absence of the object 30 and the output signal of the paddle sensor 253 indicates absence of the compression paddle 241, and the heat source 281 may be turned on in the case in which the output signal of the object sensor 251 indicates absence of the object 30 and the output signal of the paddle sensor 253 indicates presence of the compression paddle 241. In addition, after a predetermined time, the ultraviolet light generator 271 may be turned off and the heat source 281 may be turned on, or the heat source 281 may be turned off and the ultraviolet light generator 271 may be turned on.

Alternatively, when the current stage is determined to be an idle period, one of the ultraviolet light generator 271 and the heat source 281 may be first turned on, and after a predetermined time, the turned-on one may be arbitrarily turned off and the other may be turned on. Here, the predetermined time refers to a reasonable time that may be required for ultraviolet light sterilization or a reasonable time that may be required to heat the area contacting the object 30, and may be previously set or may be changed by the user.

Alternatively, only the ultraviolet light generator 271 may be turned on to perform only sterilization for a next patient, when the output signal of the object sensor 251 indicates absence of the object 30, the output signal of the paddle sensor 253 indicates absence of the compression paddle 241 and the work list having remaining X-ray imaging. At this case, the object contact area 227 may be expected to be heated by heat that may be supplied for previous X-ray imaging.

The embodiment of the X-ray imaging device 200 is not limited to the example and the power controller 231 may perform a control operation such that the ultraviolet light generator 271 and the heat source 281 may be turned on according to various algorithms.

Hereinafter, a method for controlling the X-ray imaging device according to one or more embodiments will be described.

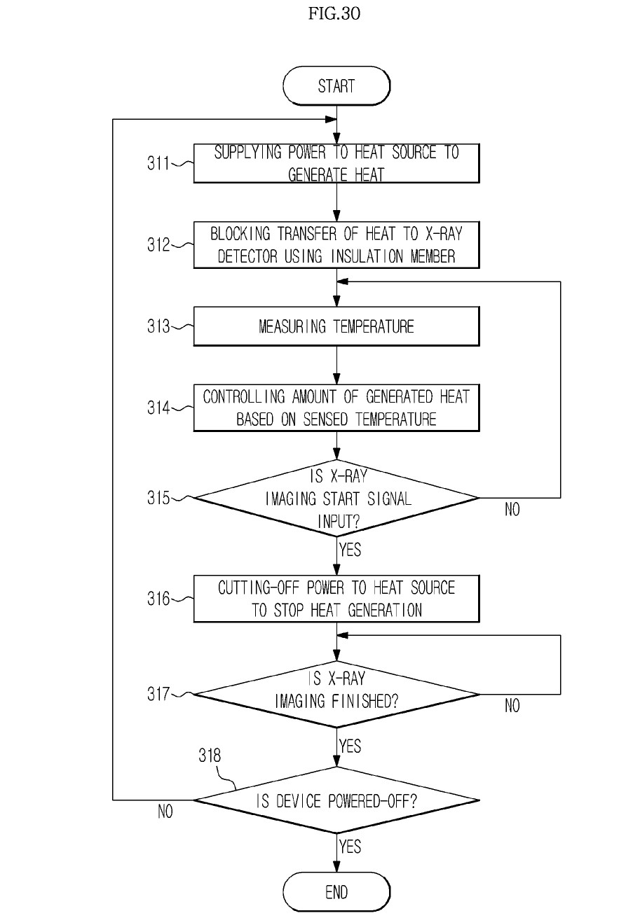

FIG. 30 is a flowchart illustrating a method for controlling an X-ray imaging device according to one or more embodiments. For example, the X-ray imaging device 100 may be employed.

Referring to FIG. 30, power may be supplied to a heat source to generate heat (311). The heat source 151 may heat an area contacting the breast 30 and thereby may allow a patient to feel warm when the breast 30 is compressed. The heat source 151 may transfer heat by radiation and heat generated by the heat source 151 may be radiant heat. For example, the heat source 151 may be implemented as an infrared lamp to generate infrared rays having a predetermined wavelength band. The heat source 151 may be mounted in a lower part of the X-ray source 110 or on a front surface of the frame 103 to heat the lower part of the compression paddle 141 and the object contact area 127.

Transfer of heat to the X-ray detector may be blocked using an insulation member (312). Although the lower part of the compression paddle 141 and the object contact area 127 are heated by the heat source 151, it may be necessary to maintain the temperature of the X-ray detector 121 at a predetermined level suitable for characteristics thereof. Accordingly, an insulation member 123 may be provided between the object contact area 127 and the X-ray detector 121 to block transfer of heat to the X-ray detector 121. The insulation member 123 may block infrared light, but may not block X-rays.

The temperature of the object contact area or the compression paddle may be sensed (313). The sensing of temperature may be carried out in real-time or on a regular cycle. A temperature sensor 125 mounted on the object contact area or compression paddle may be used for temperature sensing.

An amount of generated heat may be controlled based on the sensed temperature (314). Specifically, the amount of heat generated by the heat source 151 may be controlled so as to maintain the temperature of the area contacting the object within a predetermined range. When the sensed temperature is lower than a predetermined lower limit, the amount of heat generated by the heat source 151 may be increased and when the sensed temperature is higher than a predetermined upper limit, the amount of heat generated by the heat source 151 may be decreased or power may be cut off to the heat source 151 to stop heat generation for a predetermined time.

When X-ray imaging starts (Yes of 315), power may be cut off to the heat source and heat generation may thus be stopped (316). It may be determined that X-ray imaging starts when power is supplied to the X-ray source 110 or a user inputs a separate control instruction through the input unit 162 for X-ray imaging.

When X-ray imaging finishes (YES of 317), it may be determined whether the X-ray imaging device is powered off (318). When the X-ray imaging device is not powered off (NO of 318), power may be supplied to the heat source again to generate heat.

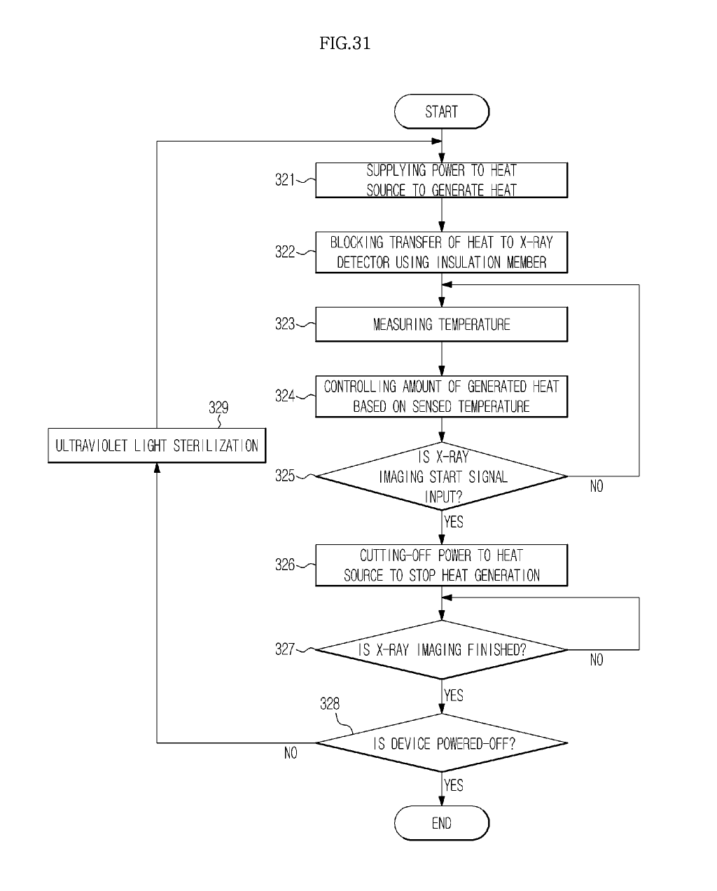

FIG. 31 is a flowchart illustrating a method for controlling an X-ray imaging device according to one or more embodiments further including ultraviolet light sterilization. For example, the X-ray imaging device 100 may be employed.

The operations of supplying power to the heat source to generate heat (321) through the determining of whether the X-ray imaging device is powered off (328) are the same as described with reference to FIG. 13 and a detailed description thereof will be thus omitted.

When X-ray imaging finishes (YES of 327) and the X-ray imaging device is not powered off (NO of 328), ultraviolet light sterilization may be performed (329). For this purpose, the ultraviolet light generator 171 may be mounted in a lower part of the X-ray source 110 or on a front surface of the frame 103 to generate ultraviolet light having a wavelength range of, for example, about 250 nm to 260 nm. Microorganisms may be destroyed and sterilization effects may thus be obtained, when ultraviolet light is emitted to the compression paddle 141 and the object contact area 127.

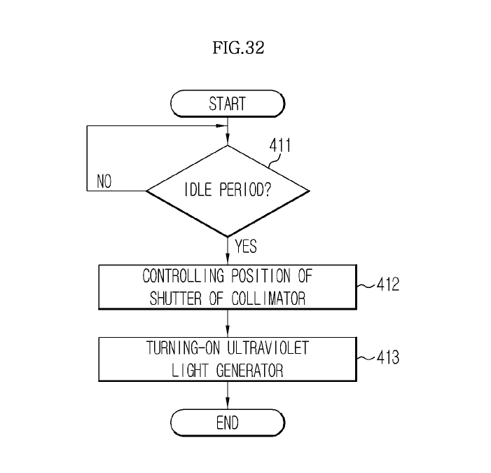

FIG. 32 is a flowchart illustrating a method of controlling an X-ray imaging device according to one or more embodiments. According to one or more embodiments, the afore-mentioned X-ray imaging device 200 may be employed.

Referring to FIG. 32, first, whether or not a current stage is an idle period may be determined (411), and position of shutters 213a of a collimator 213 may be controlled (412) when the current state corresponds to the idle period (YES of 411). For this purpose, a controller 230 may control a shutter driving unit to move shutters 213a to a position corresponding to a predetermined ultraviolet light emission area. The position corresponding to the predetermined ultraviolet light emission area may mean a position of the shutters 213a which enables position and area of an opening R formed by the shutters 213a to be equivalent to or correspond to position and area of the predetermined ultraviolet light emission area. Meanwhile, the region corresponding to an object contact area 227 may be set as a basis of the ultraviolet light emission area and may be changed by the user or the controller 230 as necessary.

After the position of the shutter 213a of the collimator 213 is controlled, the ultraviolet light generator 271 may be turned on (413) to possibly sterilize the object contact area 227. For this purpose, the controller 230 may control the power supply 272 of the ultraviolet light generation unit 270 to apply power to the ultraviolet light generator 271 and to automatically turn off the ultraviolet light generator 271 after a predetermined time. Here, the predetermined time may refer to a time required for ultraviolet light sterilization and may be previously set or be changed by a user. Alternatively, the controller 230 may turn off the ultraviolet light generator 271 when the idle period is determined to have ended. Specifically, the ultraviolet light generator 271 may be automatically turned off when an output signal of an object sensor 251 indicates presence of an object 30 or an output signal of a paddle sensor 253 indicates presence of a compression paddle 241. In addition, the ultraviolet light generator 271 may be automatically turned off upon receiving a work list for new X-ray imaging.

Meanwhile, the X-ray imaging device 200 may further include a heat source unit 280 to heat the object contact area 227. When the current state is determined to be an idle period (YES of 411), the heat source 281 may be turned on before the ultraviolet light generator 271 is turned on and, after a predetermined time, the heat source 281 may be turned off and the ultraviolet light generator 271 may be turned on. Alternatively, conversely, at a predetermined time after the ultraviolet light generator 271 is first turned on, the ultraviolet light generator 271 may be turned off and the heat source 281 may be turned on. Here, the predetermined time may refer to a time required for ultraviolet light sterilization or a time required to heat the area contacting the object 30 and may be previously set or may be changed by the user.

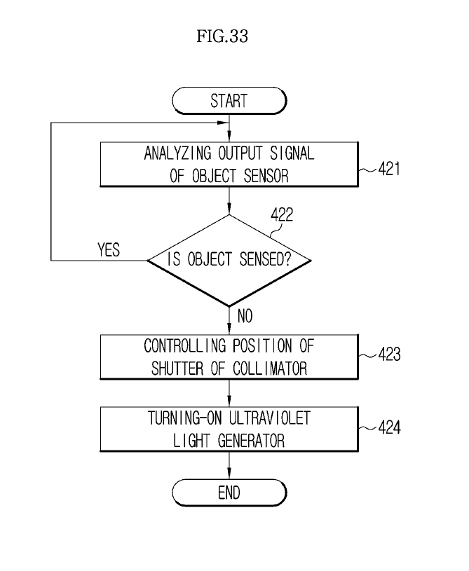

FIG. 33 is a flowchart illustrating a control process of generation of ultraviolet light according to presence of an object, in a method of controlling an X-ray imaging device according to one or more embodiments.

Referring to FIG. 33, an output signal of the object sensor 251 may be analyzed to determine whether or not the current stage is an idle period (421), and position of the shutter 213a of the collimator 213 may be controlled (423) when the output signal of the object sensor 251 indicates that an object is not sensed (NO of 422).

Here, the expression “object is not sensed” means absence of the object 30 and the absence of the object 30 means that the object 30 is not disposed on the object contact area 227 or the object 30 is not adjacent to the object contact area 227.

Specifically, the analysis of the output signal of the object sensor 251 (421) may include comparing the output signal of the object sensor 251 with a predetermined reference value and the object may be determined to be not sensed when the output signal of the object sensor 251 is equivalent to or higher than the predetermined reference value.

After control of the position of the shutter 213a of the collimator 213, the ultraviolet light generator 271 may be turned on (424) to possibly sterilize the object contact area 227.

After a predetermined time, the ultraviolet light generator 271 may be automatically turned off. Here, the predetermined time refers to a time that may be required for ultraviolet light sterilization and may be previously set or may be changed by a user. In addition, the ultraviolet light generator 271 may be turned off when the idle period is determined to have ended and a description thereof has been given above.

Meanwhile, in the case in which the X-ray imaging device 200 further includes a heat source unit 280 to heat the object contact area 227, when an object is not sensed (NO of 422), the heat source 281 may be turned on before the ultraviolet light generator 271 is turned on and, after a predetermined time, the heat source 281 may be turned off and the ultraviolet light generator 271 may be turned on. Alternatively, conversely, at a predetermined time after the ultraviolet light generator 271 is first turned on, the ultraviolet light generator 271 may be turned off and the heat source 281 may be turned on. Here, the predetermined time refers to a time required for ultraviolet light sterilization or a time required to heat the object contact area 227 and may be previously set or may be changed by the user.

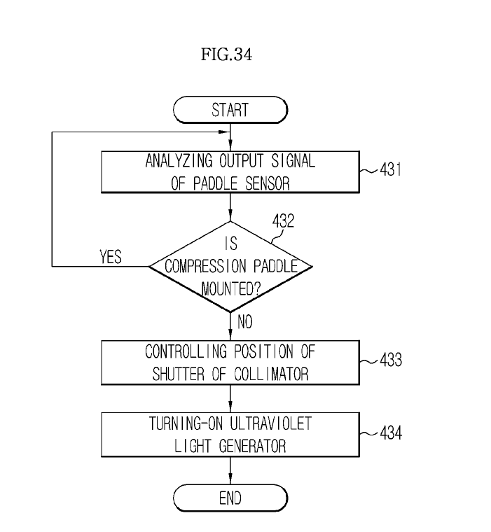

FIG. 34 is a flowchart illustrating a control process of generation of ultraviolet light according to presence of a compression paddle, in a method of controlling an X-ray imaging device according to one or more embodiments.

Referring to FIG. 34, an output signal of the paddle sensor 253 may be analyzed to determine whether or not the current stage is an idle period (431), and position of the shutter 213a of the collimator 213 may be controlled (433) when the output signal of the paddle sensor 253 indicates that a compression paddle is not mounted (NO of 432).

Here, the expression “compression paddle is not mounted” means absence of the compression paddle 241 and the absence of the compression paddle 241 means that the compression paddle 241 is not mounted in the frame 203.

After control of the position of the shutter 213a of the collimator 213, the ultraviolet light generator 271 may be turned on (444) to sterilize the object contact area 227.

After a predetermined time, the ultraviolet light generator 271 may be automatically turned off. Here, the predetermined time refers to a time required for ultraviolet light sterilization and may be previously set or be changed by a user. In addition, the ultraviolet light generator 271 may be turned off when the idle period is determined to have ended and a description thereof has been given above.

Meanwhile, in the case in which the X-ray imaging device 200 further includes a heat source unit 280 to heat the object contact area 227, when the compression paddle 241 is not mounted (NO of 432), the heat source 281 may be first turned on and, after a predetermined time, the heat source 281 may be turned off and the ultraviolet light generator 271 may be turned on. Alternatively, conversely, at a predetermined time after the ultraviolet light generator 271 is first turned on, the ultraviolet light generator 271 may be turned off and the heat source 281 may be turned on. Here, the predetermined time refers to a time required for ultraviolet light sterilization or a time required to heat the object contact area 227 and may be previously set or may be changed by the user.

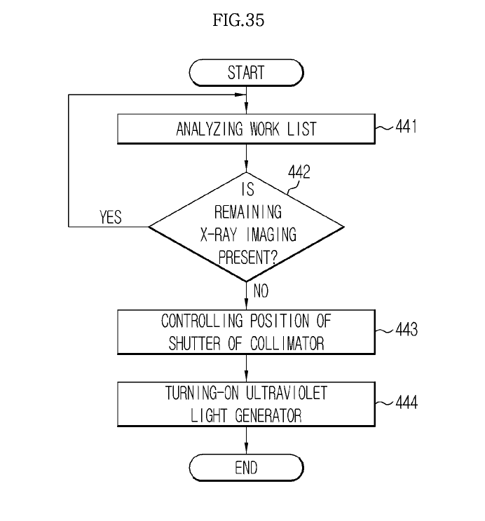

FIG. 35 is a flowchart illustrating a control process of generation of ultraviolet light through analysis of a work list, in a method of controlling an X-ray imaging device according to one or more embodiments.

Referring to FIG. 35, a work list may be analyzed (441), the current state may be determined to be an idle period when remaining X-ray imaging is not present (NO of 442) and position of the shutter 213a of the collimator 213 may be controlled (443).

In addition, the controller 230 may control the power supply 272 to apply power to the ultraviolet light generator 271 and to turn on the ultraviolet light generator 271 (444).

After a predetermined time, the ultraviolet light generator 271 may be automatically turned off. Here, the predetermined time refers to a time required for ultraviolet light sterilization and may be previously set or be changed by a user. In addition, the ultraviolet light generator 271 may be turned off when the idle period is determined to have ended and a description thereof has been given above.

Meanwhile, in the case in which the X-ray imaging device 200 further includes the heat source unit 280 to heat the object contact area 227, when remaining X-ray imaging is not present (NO of 442), the heat source 281 may be first turned on and, after a predetermined time, the heat source 281 may be turned off and the ultraviolet light generator 271 may be turned on. Alternatively, conversely, at a predetermined time after the ultraviolet light generator 271 may be first turned on, the ultraviolet light generator 271 may be turned off and the heat source 281 may be turned on. Here, the predetermined time refers to a time required for ultraviolet light sterilization or a time required to heat the object contact area 227 and may be previously set or may be changed by the user.

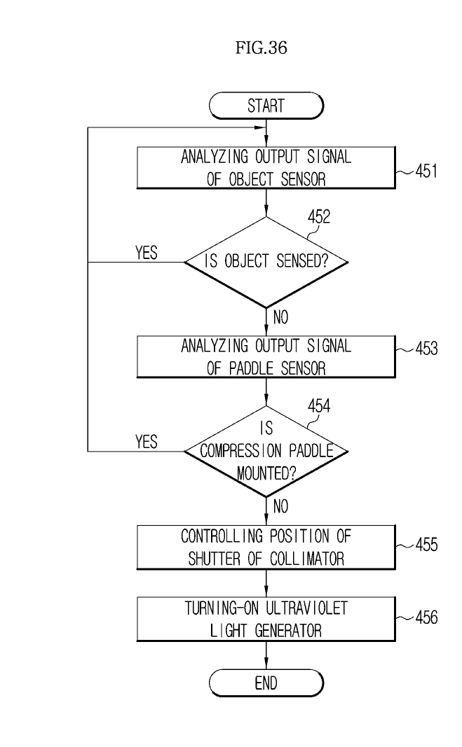

FIG. 36 is a flowchart illustrating a control process of generation of ultraviolet light according to presence of an object and a compression paddle, in a method of controlling an X-ray imaging device according to one or more embodiments.

Referring to FIG. 36, an output signal of the object sensor 251 may be analyzed (451) and an output signal of the paddle sensor 253 may be analyzed (453) when the output signal of the object sensor 251 indicates that an object is not sensed (NO of 422).

Position of the shutter 213a of the collimator 213 may be controlled (455) when the output signal of the paddle sensor 253 indicates that the compression paddle 241 is not mounted (NO of 454). In addition, an ultraviolet light generator 271 may be turned on (456) to possibly sterilize an object contact area 227.

After a predetermined time, the ultraviolet light generator 271 may be automatically turned off again. Here, the predetermined time refers to a time that may be required for ultraviolet light sterilization and may be previously set or be changed by a user. In addition, the ultraviolet light generator 271 may be turned off when the idle period is determined to have ended, and a description thereof has been given above.

Meanwhile, in the case in which the X-ray imaging device 200 further includes the heat source unit 280 to heat the object contact area 227, when an object is not sensed (NO of 452) and the compression paddle 241 is not mounted (NO of 454), the heat source 281 may be first turned on and, after a predetermined time, the heat source 281 may be turned off and the ultraviolet light generator 271 may be turned on (456). Alternatively, conversely, at a predetermined time after the ultraviolet light generator 271 is first turned on (456), the ultraviolet light generator 271 may be turned off and the heat source 281 may be turned on. Here, the predetermined time refers to a time required for ultraviolet light sterilization or a time required to heat the object contact area 227 and may be previously set or may be changed by the user.

Alternatively, the object contact area 227 may be heated by turning on only the heat source 281 while not turning on the ultraviolet light generator 271, when object is not sensed (NO of 452) and the compression paddle 241 is mounted (YES of 454), because heating of the object contact area 227 may be possible although the compression paddle 241 is mounted.

For convenience of description, the output signal of the object sensor 251 may be first analyzed in the example shown in FIG. 36. However, the output signal of the paddle sensor 253 may be first analyzed, and the output signal of the object sensor 251 and the output signal of the paddle sensor 253 may be simultaneously analyzed. There is no limitation as to analysis order so long as ultraviolet light sterilization may be performed when both the object 30 and the compression paddle 241 are not present.

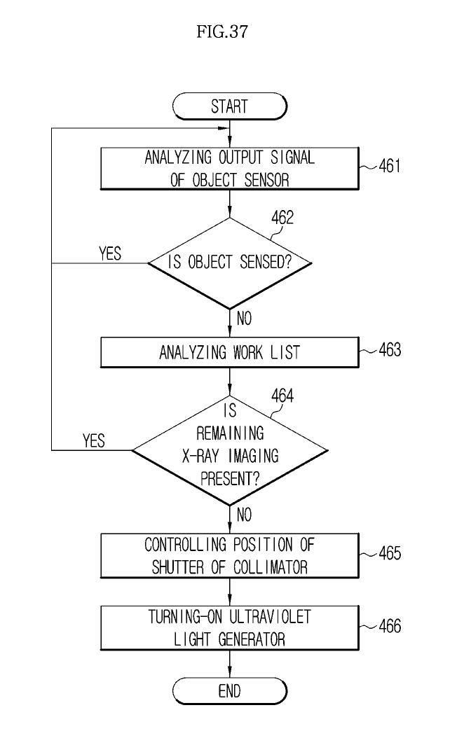

FIG. 37 is a flowchart illustrating a control process of generation of ultraviolet light based on presence of an object and a work list, in a method of controlling an X-ray imaging device according to one or more embodiments.

Referring to FIG. 37, an output signal of the object sensor 251 may be analyzed (461) and a work list may be analyzed (463) when the output signal of the object sensor 251 indicates that an object is not sensed (NO of 462).

When remaining X-ray imaging is not present in the work list (464), the current state may be determined to be an idle period and position of the shutter 213a of the collimator 213 may be controlled (465), and the ultraviolet light generator 271 may be turned on (466) to possibly sterilize the object contact area 227.

After a predetermined time, the ultraviolet light generator 271 may automatically be turned off again. Here, the predetermined time refers to a time required for ultraviolet light sterilization and may be previously set or be changed by a user. In addition, the ultraviolet light generator 271 may be turned off when the idle period is determined to have ended and a description thereof has been given above.

Meanwhile, in the case in which the X-ray imaging device 200 further includes the heat source unit 280 to heat the object contact area 227, when the object is not sensed (NO of 462) and remaining X-ray imaging is not present (NO of 464), the heat source 281 may be first turned on and, after a predetermined time, the heat source 281 may be turned off and the ultraviolet light generator 271 may be turned on (466). Alternatively, conversely, at a predetermined time after the ultraviolet light generator 271 may be first turned on (466), the ultraviolet light generator 271 may be turned off and the heat source 281 may be turned on. Here, the predetermined time refers to a time required for ultraviolet light sterilization or a time required to heat the object contact area 227 and may be previously set or may be changed by the user.

For convenience of description, the output signal of the object sensor 251 may be first analyzed in the example shown in FIG. 37. However, the work list may be first analyzed, and the output signal of the object sensor 251 and the work list may be simultaneously analyzed. There is no limitation as to analysis order so long as ultraviolet light sterilization is performed when both the object 30 and remaining X-ray imaging are not present.

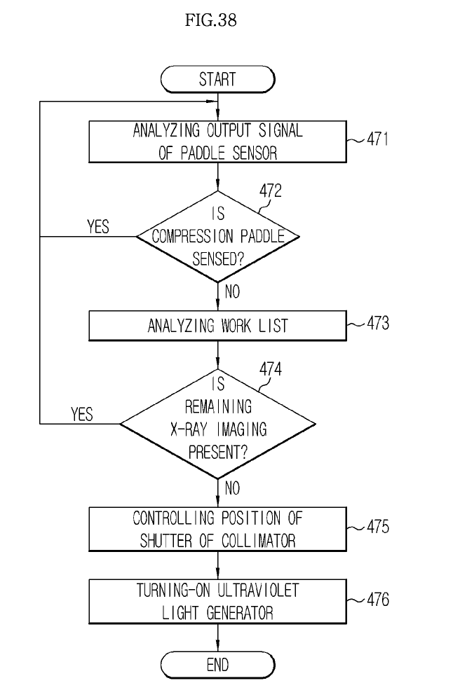

FIG. 38 is a flowchart illustrating a control process of generation of ultraviolet light based on presence of a compression paddle and a work list, in a method of controlling an X-ray imaging device according to one or more embodiments.

Referring to FIG. 38, an output signal of the paddle sensor 253 may be analyzed (471) and a work list may be analyzed (473) when the output signal of the paddle sensor 253 indicates that a compression paddle 241 is not mounted in a flame 203 (NO of 472).

When remaining X-ray imaging is not present in the work list (YES of 474), position of the shutter 213a of the collimator 213 may be controlled (475), and the ultraviolet light generator 271 may be turned on (476) to possibly sterilize the object contact area 227.

After a predetermined time, the ultraviolet light generator 271 may be automatically turned off again. Here, the predetermined time refers to a time required for ultraviolet light sterilization and may be previously set or be changed by a user. In addition, the ultraviolet light generator 271 may be turned off when the idle period is determined to have ended, and a description thereof has been given above.

Meanwhile, in the case in which the X-ray imaging device 200 further includes the heat source unit 280 to heat the object contact area 227, when the compression paddle 241 is not mounted (NO of 472), the heat source 281 may be first turned on and, after a predetermined time, the heat source 281 may be turned off and the ultraviolet light generator 271 may be turned on (476). Alternatively, conversely, at a predetermined time after the ultraviolet light generator 271 is first turned on, the ultraviolet light generator 271 may be turned off and the heat source 281 may be turned on. Here, the predetermined time refers to a time required for ultraviolet light sterilization or a time required to heat the object contact area 227 and may be previously set or may be changed by the user.

For convenience of description, the output signal of the paddle sensor 253 may be first analyzed in the example shown in FIG. 38. However, the work list may be first analyzed, and the output signal of the paddle sensor 253 and the work list may be simultaneously analyzed. There is no limitation as to analysis order so long as ultraviolet light sterilization is performed when both the compression paddle 241 and remaining X-ray imaging are not present.

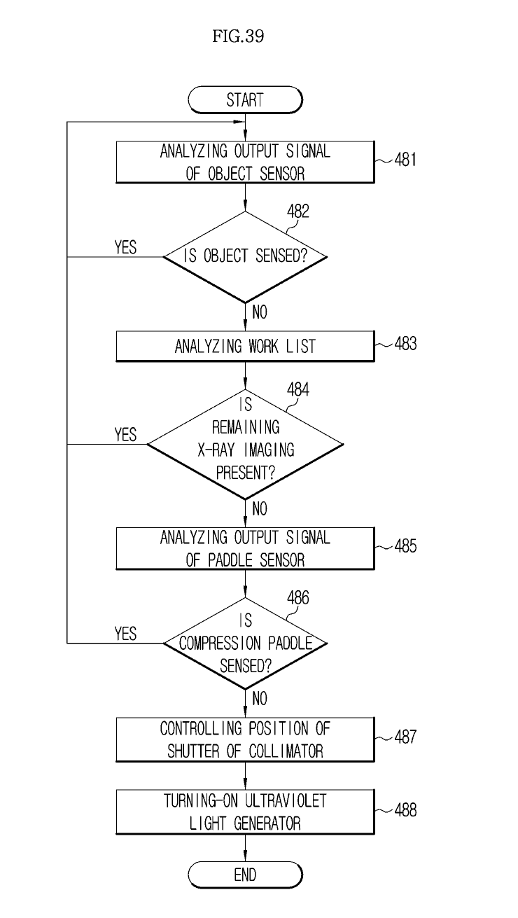

FIG. 39 is a flowchart illustrating a control process of generation of ultraviolet light based on presence of an object and a compression paddle and a work list, in the method of controlling an X-ray imaging device according to one or more embodiments.

Referring to FIG. 39, an output signal of the object sensor 251 may be analyzed (481) and a work list may be analyzed (483) when the output signal of the object sensor 251 indicates that an object is not sensed (NO of 482).

An output signal of the paddle sensor 253 may be analyzed (485) when remaining X- ray imaging is not present in the work list (YES of 484).

Position of the shutter 213a of the collimator 213 may be controlled (487) when the output signal of the paddle sensor 253 indicates that the compression paddle 241 is not mounted (NO of 486) and the ultraviolet light generator 271 may be turned on (488) to possibly sterilize the object contact area 227.

After a predetermined time, the ultraviolet light generator 271 may be automatically turned off again. Here, the predetermined time refers to a time required for ultraviolet light sterilization and may be previously set or be changed by a user. In addition, the ultraviolet light generator 271 may be turned off when the idle period is determined to have ended and a description thereof has been given above.

Meanwhile, in the case in which the X-ray imaging device 200 further includes the heat source unit 280 to heat the object contact area 227, when the object is not sensed (NO of 482), remaining X-ray imaging is not present (NO of 484), and the compression paddle 241 is not mounted (YES of 486), the heat source 281 may be first turned on and, after a predetermined time, the heat source 281 may be turned off and the ultraviolet light generator 271 may be turned on (488). Alternatively, conversely, at a predetermined time after the ultraviolet light generator 271 is first turned on (488), the ultraviolet light generator 271 may be turned off and the heat source 281 may be turned on. Here, the predetermined time refers to a time required for ultraviolet light sterilization or a time required to heat the object contact area 227 and may be previously set or may be changed by the user.

Alternatively, the object contact area 227 may be heated by only turning on the heat source 281 while not turning on the ultraviolet light generator 271, when an object is not sensed (NO of 482), remaining X-ray imaging is not present (NO of 484) and the compression paddle 241 is mounted (YES of 486), because heating of the object contact area 227 may be possible although the compression paddle 241 is mounted.

For convenience of description, the output signal of the object sensor 251, the work list, and the output signal of the paddle sensor 253 may be sequentially analyzed in this order in the example shown in FIG. 39. However, there is no limitation as to analysis order, and a part or an entirety of the output signal of the object sensor 251, the output signal of the paddle sensor 253 and the work list may be simultaneously analyzed.

As apparent from the fore-going, one or more embodiments provide an X-ray imaging device and a method for controlling the same which may generate radiant heat to elevate a temperature of an area of the X-ray imaging device contacting the breast and thereby reduce patient discomfort, and may automatically control a temperature to possibly maintain the temperature without additional operation.

Furthermore, the X-ray imaging device may generate ultraviolet light during a rest period in which X-ray imaging is not performed and may sterilize the area contacting the object with the ultraviolet light to possibly sanitize the area contacting the object.

In one or more embodiments, any apparatus, system, element, or interpretable unit descriptions herein include one or more hardware devices or hardware processing elements. For example, in one or more embodiments, any described apparatus, system, element, retriever, pre or post-processing elements, tracker, detector, encoder, decoder, etc., may further include one or more memories and/or processing elements, and any hardware input/output transmission devices, or represent operating portions/aspects of one or more respective processing elements or devices. Further, the term apparatus should be considered synonymous with elements of a physical system, not limited to a single device or enclosure or all described elements embodied in single respective enclosures in all embodiments, but rather, depending on embodiment, is open to being embodied together or separately in differing enclosures and/or locations through differing hardware elements.

In addition to the above described embodiments, embodiments can also be implemented through computer readable code/instructions in/on a non-transitory medium, e.g., a computer readable medium, to control at least one processing device, such as a processor or computer, to implement any above described embodiment. The medium can correspond to any defined, measurable, and tangible structure permitting the storing and/or transmission of the computer readable code.

The media may also include, e.g., in combination with the computer readable code, data files, data structures, and the like. One or more embodiments of computer-readable media include: magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. Computer readable code may include both machine code, such as produced by a compiler, and files containing higher level code that may be executed by the computer using an interpreter, for example. The media may also be any defined, measurable, and tangible distributed network, so that the computer readable code is stored and executed in a distributed fashion. Still further, as only an example, the processing element could include a processor or a computer processor, and processing elements may be distributed and/or included in a single device.

The computer-readable media may also be embodied in at least one application specific integrated circuit (ASIC) or Field Programmable Gate Array (FPGA), as only examples, which execute (e.g., processes like a processor) program instructions.

While aspects of the present invention have been particularly shown and described with reference to differing embodiments thereof, it should be understood that these embodiments should be considered in a descriptive sense only and not for purposes of limitation. Descriptions of features or aspects within each embodiment should typically be considered as available for other similar features or aspects in the remaining embodiments. Suitable results may equally be achieved if the described techniques are performed in a different order and/or if components in a described system, architecture, device, or circuit are combined in a different manner and/or replaced or supplemented by other components or their equivalents.

Thus, although a few embodiments have been shown and described, with additional embodiments being equally available, it would be appreciated by those skilled in the art that changes may be made in these embodiments without departing from the principles and spirit of the invention, the scope of which is defined in the claims and their equivalents.

What is claimed is:

1. An X-ray imaging device of producing an X-ray image comprising:
an X-ray source to generate X-rays and emit the X-rays to an object;
an X-ray detector to detect X-rays transmitted through the object and to convert the X-rays into an electrical signal;
an object contact area mounted in an upper part of the X-ray detector, the object contact area contacting the object;
a compression paddle to compress the object disposed on the object contact area;
a heat source to generate heat and transfer the heat to at least one of the object contact area and the compression paddle; and
an insulation member formed between the object contact area and the X-ray detector, the insulation member blocking transfer of heat to the X-ray detector.

2. The X-ray imaging device according to claim 1, wherein the heat source is mounted in a lower part of the X-ray source.

3. The X-ray imaging device according to claim 1, wherein the insulation member comprises a film to block transfer of heat.

4. The X-ray imaging device according to claim 1, wherein the insulation member comprises a coating layer comprising a material blocking transfer of heat, the coating layer being formed on the X-ray detector.

5. The X-ray imaging device according to claim 1, wherein the insulation member does not block transmission of X-rays.

6. The X-ray imaging device according to claim 1, further comprising a temperature sensor to sense a temperature of the object contact area,
wherein the temperature sensor is disposed outside of the X-ray detector.

7. The X-ray imaging device according to claim 6, further comprising a controller to control an amount of heat generated by the heat source, based on the temperature sensed by the temperature sensor.

8. The X-ray imaging device according to claim 7, wherein the controller decreases an amount of heat generated by the heat source when the temperature sensed by the temperature sensor is higher than a predetermined upper limit and increases the amount of heat generated by the heat source when the temperature sensed by the temperature sensor is lower than a predetermined lower limit.

9. The X-ray imaging device according to claim 7, wherein the controller cuts off power to the heat source to stop heat generation when X-ray imaging starts.

10. The X-ray imaging device according to claim 7, further comprising an ultraviolet light generator to emit ultraviolet light to at least one of the object contact area and the compression paddle.

11. The X-ray imaging device according to claim 10, wherein the controller turns off the ultraviolet light generator when X-ray imaging starts, and turns on the ultraviolet light generator when X-ray imaging finishes.

12. The X-ray imaging device according to claim 1, wherein the heat source generates radiant heat, wherein the radiant heat comprises infrared light.

13. The X-ray imaging device according to claim 1, wherein the object contact area comprises carbon.

14. The X-ray imaging device according to claim 1, wherein the insulation member blocks infrared light having a wavelength range of about 0.75 μm to about 1 mm and does not block X-rays having a wavelength range of about 0.001 nm to about 10 nm.

15. A method of controlling an X-ray imaging device to produce an X-ray image comprising:
supplying power to a heat source mounted on the X-ray imaging device to generate heat;
blocking transfer of the generated heat to an X-ray detector using an insulation member disposed between the heat source and the X-ray detector; and
cutting-off power to the heat source to stop heat generation when X-ray imaging starts.

16. The method according to claim 15, wherein the heat source is mounted at a position enabling transfer of heat to at least one of a compression paddle and an object contact area mounted on the X-ray imaging device contacting the object.

17. The method according to claim 16, wherein the heat source transfers heat by radiation.

18. The method according to claim 16, further comprising sensing a temperature of at least one of the compression paddle and the object contact area.

19. The method according to claim 18, further comprising controlling an amount of heat generated by the heat source, based on the sensed temperature.

20. The method according to claim 19, wherein the controlling comprises increasing the amount of heat generated by the heat source when the sensed temperature is lower than a predetermined lower limit and decreasing the amount of heat generated by the heat source when the sensed temperature is higher than a predetermined upper limit.

21. The method according to claim 20, further comprising emitting ultraviolet light to at least one of the compression paddle and the object contact area when X-ray imaging finishes.
